(19) United States
(12) Patent Application Publication
(10) Pub. No.: US 2011/0294782 A1
(43) Pub. Date: Dec. 1, 2011

(54) SMALL MOLECULE PAK INHIBITORS

(75) Inventors: Susumu Tonegawa, Chestnut Hill, MA (US); Mansuo L. Hayashi, Winchester, MA (US); Mazen Karaman, San Diego, CA (US); Patrick P. Zarrinkar, San Diego, CA (US); Daniel K. Treiber, San Diego, CA (US)

(73) Assignee: Massachusetts Institute of Technology, Boston, MA (US)

(21) Appl. No.: 12/514,290

(22) PCT Filed: Nov. 9, 2007
(86) PCT No.: PCT/US2007/023591

§ 371 Date: Feb. 1, 2010

Related U.S. Application Data
(60) Provisional application No. 60/858,108, filed on Nov. 10, 2006.

(51) Int. Cl. A61K 31/553; A61K 31/496; A61P 25/00; A61K 31/4196; A61K 31/5377; A61K 31/404; A61K 31/454; A61K 31/4706

(52) U.S. Cl. 514/211.08; 514/326; 514/254.06; 514/313; 514/383; 514/235.2; 514/414; 514/252.19

(57) ABSTRACT

The present invention provides methods for treating fragile X syndrome and/or other neurodevelopmental disorders by administering small molecule p21-activated kinase (PAK) inhibitors to a patient suffering from, susceptible to, and/or exhibiting one or more symptoms of FXS and/or other neurodevelopmental disorders. The present invention provides specific small molecule PAK inhibitors and pharmaceutical compositions comprising small molecule PAK inhibitors.

### RELATED APPLICATIONS

This application claims priority under 35 U.S.C. §119(e) to U.S. provisional patent application, U.S. Ser. No. 60/858,108, filed Nov. 10, 2006 (“the 108 application”). The entire contents of the '108 application are incorporated herein by reference.

### GOVERNMENT SUPPORT

The United States Government has provided grant support utilized in the development of the present invention. In particular, the National Institutes of Health (contract number MH78821) and the National Institute of Mental Health Center (contract number MH58880) have supported development of this invention. The United States Government has certain rights in the invention.

### BACKGROUND OF THE INVENTION

Fragile X syndrome (FXS) is the most commonly inherited form of mental retardation, with symptoms of hyperactivity, stereotypy, anxiety, seizure, impaired social behavior, and/or cognitive delay. FXS results from the loss of expression of the fragile X mental retardation 1 (FMR1) gene, which encodes the fragile X mental retardation protein (FMRP). FMR1 knockout (FMR1 KO) mice and FXS patients show similar behavioral phenotypes, as well as similar abnormalities in synaptic morphology in the brain (O'Donnell et al., 2002, Annu. Rev. Neurosci., 25:315; incorporated herein by reference). Their brains have more dendritic spines and/or a higher proportion of longer and/or thinner spines compared to normal individuals (Hinton et al., 1991, Am. J. Med. Genet., 41:289; Comery et al., 1997, Proc. Natl. Acad. Sci., USA, 94:5401; and Irwin et al., 2001, Am. J. Med. Genet., 98:161; all of which are incorporated herein by reference). Dendritic spines are the protrusions from dendritic shafts that serve as postsynaptic sites of the majority of excitatory synapses in mammals. Correlated with altered spine morphology, FMR1 KO mice display abnormal synaptic function, including enhanced long-term depression (LTD) mediated by metabotropic glutamate receptor in the hippocampus and/or impaired long-term potentiation (LTP) in the cortex, compared to wild type mice (Huber et al., 2002, Neuropharmacology, 37:571; and Li et al., 2002, Mol. Cell Neurosci., 19:138; both of which are incorporated herein by reference). Thus, these findings demonstrate that FMRP functions in regulating spine morphology, synaptic function, and/or animal behavior.

FMRP is a selective RNA-binding protein that associates with polyribosomes (Corbin et al., 1997, Hum. Mol. Genet., 6:1465; and Stefani et al., 2004, J. Neurosci., 24:7272; both of which are incorporated herein by reference) and with Argonaute2 (AGO2) and Dicer, two members of RISC, a complex that is required for RNAi-mediated gene silencing (Ishizuka et al., 2002, Genes Dev., 16:2497; incorporated herein by reference). FMRP is thought to regulate synaptic morphology and/or function because of its ability to repress translation of its RNA binding partners (Laggerbauer et al., 2001, Hum. Mol. Genet., 10:329; Li et al., 2001, Nucleic Acids Res., 29:2276; and Mazroui et al., 2002, Hum. Mol. Genet., 11:3007; all of which are incorporated herein by reference), perhaps using an RNAi-mediated mechanism. Some of these RNAs encode proteins that are involved in synaptic morphology and/or function, such as Rac1, microtubule-associated protein 1B, activity-regulated cytoskeleton-associated protein, and alpha-calcium/calmodulin-dependent protein kinase II (Zhang et al., 2001, Cell, 107:591; Lee et al., 2003, Development, 130:5543; and Zalfa et al., 2003, Cell, 112:317; all of which are incorporated herein by reference).

### SUMMARY OF THE INVENTION

The present invention provides systems, including methods, reagents, and/or compositions, for treating fragile X syndrome (FXS) and/or other neurodevelopmental disorders comprising administering a small molecule PAK inhibitor to a subject susceptible to, suffering from, and/or exhibiting symptoms of FXS and/or other neurodevelopmental disorder.

In accordance with some embodiments, a pharmaceutical composition is provided comprising a small molecule PAK inhibitor and a pharmaceutically acceptable excipient.

The present invention provides specific small molecule PAK inhibitors, including BMS-387032; SNS-032; CHI4-258; TKI-258; EKB-569; JNJ-7706621; PKC-412; staurosporine; SU-14813; sunitinib; VX-680; MK-0457; combinations thereof; and/or analogs or derivatives thereof. Also described herein are small molecule PAK inhibitors that are selective for a particular PAK; in one embodiment the small molecule PAK inhibitor is selective for a PAK selected from the group consisting of PAK1, PAK2, PAK3, PAK4, PAK6, and PAK7. The present invention provides pharmaceutical compositions comprising BMS-387032; SNS-032; CHI4-258; TKI-258; EKB-569; JNJ-7706621; PKC-412; staurosporine; SU-14813; sunitinib; VX-680; MK-0457; combinations thereof; and/or analogs or derivatives thereof and a pharmaceutically-acceptable excipient. The present invention provides methods of treating FXS and/or other neurodevelopmental disorders comprising administering BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof to a patient susceptible to, suffering from, and/or exhibiting symptoms of FXS and/or other neurodevelopmental disorder.

This application refers to various patent publications and non-patent publications, the contents of all of which are incorporated herein by reference.

### BRIEF DESCRIPTION OF THE DRAWING

FIG. 1. Representative FMR and FXR amino acid sequences. FIG. 1A shows an alignment of representative examples of Drosophila melanogaster FMR1 (dFMR1; GenBank AAG22045), human FMR1 (hFMR1; GenBank AAB18829), human FXR1 (hFXR1; GenBank AAC50155), and human FXR2 (hFXR2; GenBank AAC50292) amino acid sequences. Dark gray boxes mark identical amino acids, and light gray boxes denote conservative amino acid substitutions. Insertions are denoted by a dash. The FMR1/FXR interaction domain is depicted. “KH1” and “KH2”: KH domains; “NES”: nuclear export signal; “RGG”: domain that mediates protein-protein interactions, RNA-binding, and/or may be methylated. FIG. 1A modified from Wan et al., 2000, Mol. Cell. Biol., 20:8536 (incorporated herein by reference). FIG. 1B shows an amino acid sequence for mouse FMR1 (GenBank NM_{—}008031).

FIG. 2. Representative human PAK1, PAK2, and PAK3 amino acid sequences. Shown is an alignment of representative examples of human PAK1 (GenBank AAA65441), human PAK2 (GenBank AAA65442), and human PAK3 (GenBank AAC36097) amino acid sequences. Asterisks below the sequences mark identical amino acids, and dots below the sequences denote conservative amino acid substitutions. Insertions are denoted by a dash. Black boxes mark proline-rich regions containing putative SH3-binding PXXP motifs. The open box marks the noncanonical PIX binding site. Gray boxes indicate highly charged basic or acidic tracts. The dark overhead lines indicate the homodimerization domain (amino acids 78-87), CRIB motif (amino acids 75-90), p21-binding domain (PBD; amino acids 67-113), and autoinhibitory switch domain (amino acids 83-139) for PAK1. Diagnostic kinase motifs in the catalytic domain are boxed and numbered per convention. Figure modified from Bokoch et al., 2003, Annu. Rev. Biochem., 72:743 (incorporated herein by reference).

FIG. 3. Representative human PAK4, PAK5, PAK6, and PAK7 amino acid sequences. Shown is an alignment of representative examples of human PAK4 (GenBank NP_{—}005875), human PAK5 (GenBank CAC18720), human PAK6 (GenBank NP_{—}064553), and human PAK7 (GenBank Q9P286) amino acid sequences. Asterisks below the sequences mark identical amino acids, and dots below the sequences denote conservative amino acid substitutions. Insertions are denoted by a dash.

FIG. 4. Representative human PAK4, PAK5, and PAK6 amino acid sequences. Shown is an alignment of representative examples of human PAK4 (GenBank CAA09820), human PAK5 (GenBank BAA94194), and human PAK6 (GenBank AAF82800) amino acid sequences. Black boxes mark identical amino acids. Insertions are denoted by a dash. Diagnostic kinase motifs in the catalytic domain are boxed and numbered per convention. Figure modified from Dan et al., 2002, Mol. Cell. Biol., 22:567 (incorporated herein by reference).

FIG. 5. Representative Caenorhabditis elegans, Drosophila melanogaster, and rat PAK1 amino acid sequences. Shown is an alignment of representative examples of PAK1 amino acid sequences from C. elegans (CEPAK; GenBank BAA11844), D. melanogaster, (DPAK; GenBank AAC47094), and rat (PAK; GenBank AAB95646). Black boxes mark identical amino acids. Insertions are denoted by a dash. The N-terminal box marks the p21-binding domain, and the C-terminal box denotes the C-terminal kinase domain. Figure modified from Chen et al., 1996, J. Biol. Chem., 271:26362 (incorporated herein by reference).

FIG. 6. Golgi analysis. Representative dendritic segments of layer II/III pyramidal neurons from wild-type (WT; n=20 neurons, 2 mice), dnPAK TG mice (n=30 neurons, 3 mice), FMR1 KO mice (n=20 neurons, 2 mice), and double mutant dnPAK TG; FMR1 KO mice (dMT; n=40 neurons, 4 mice). On each primary apical dendritic branch, ten consecutive 10 μm-long dendritic segments were analyzed to quantify spine density per 10 μm-long dendritic segment (FIG. 7) and mean spine density (FIG. 8).

FIG. 7. Quantification of spine density. On each primary apical dendritic branch, ten consecutive 10 μm-long dendritic segments were analyzed to quantify spine density per 10 μm-long dendritic segment. Spine density in dMTs was comparable to wild-type controls in all dendritic segments except segment 7 and 8 (p>0.05 in segments 1-6, 9, and 10; p<0.01 in segments 7 and 8).

FIG. 8. Quantification of mean spine density. (A) On each primary apical dendritic branch, ten consecutive 10 μm-long dendritic segments were analyzed to quantify mean spine density per 10 μm-long dendritic segment. Mean spine density in dMTs (1.28±0.02) was significantly lower than that in FMR1 KO mice (1.60±0.02; p<0.001) and significantly higher than that in dnPAKTG mice (1.06±0.01; p<0.001). ANOVA, p<0.0001. ***: p<0.001.

FIG. 9. Quantification of spine length. FMR1 KO neurons (444 spines) exhibited a significant shift in the overall spine distribution towards spines of longer length compared to wild-type neurons (406 spines; Kolmogorov-Smirnov test: p<0.05), while dnPAK TG neurons (630 spines) exhibited the opposite shift to shorter spines (p<0.01). In contrast, spine length distribution in dMT neurons (785 spines) overlapped well with wild-type neurons and is significantly different from FMR1 KO neurons (p<0.01).

FIG. 10. PAK inhibition rescues reduced cortical LTP in FMR1 KO mice. (A) Input-output curves plotting the changes in field excitatory post-synaptic potential (fEPSP) amplitude and their corresponding presynaptic stimulus intensity in wild-type (n=45 slices, 16 mice), dnPAKTG (n=30 slices, 10 mice), FMR1 KO (n=57 slices, 19 mice) and dMT mice (n=24 slices, 8 mice). (B) Cortical LTP induced by TBS was enhanced in dnPAK TG (n=13 slices, 11 mice), but reduced in FMR1 KO (n=17 slices, 11 mice), relative to wild-type controls (n=17 slices, 11 mice; for responses at 55 minutes post stimulation, ANOVA, p<0.05; for both dnPAK TG versus WT and FMR1 KO versus WT, p<0.04). By contrast, the magnitude of LTP was indistinguishable between dMT slices (n=13 slices, 9 mice) and wild-type controls (p>0.05 for responses at 55 minutes post stimulation). An overlay of representative field potential traces taken during baseline of recording and at 55 minutes post stimulation is shown for each genotype.

FIG. 11. Open field test. Mice of different genotypes were subjected to the open field test according to standard procedures. Each mouse ran for 10 minutes in an activity monitor chamber. Open field activity was detected by photobeam breaks and analyzed by VersaMax software. Activities measured were the amount of time the mouse spent in the center of the field, the number of times the mouse exhibited repetitive behaviors (“stereotypy”), and the total distance traveled by the mouse. Genotypes are as follows: (1) wild-type (n=10 mice); (2) dnPAK TG (n=10 mice); (3) FMR1 KO (n=11 mice); and (4) dnPAK TG; FMR1 KO (“dMT mice” n=11 mice). “n.s.”: not statistically different. *: p<0.05; ***: p<0.001. (A) FMR1 KO traveled a longer distance compared to wild-type mice (ANOVA, p<0.01. WT: 15.29±0.92 m; FMR1 KO: 20.99±1.10 m, p<0.001). (B) FMR1 KO exhibited a higher number of repetitive behaviors than wild-type mice (stereotypy counts: ANOVA, p<0.05. WT: 1636±119; FMR1 KO: 2049±125, p<0.05). (C) FMR1 KO stayed a longer period of time in the center of the open field than wild-type mice (ANOVA, p<0.001. WT: 79.8±8.5 sec; FMR1 KO: 143.1±12.0 sec, p<0.001). In all of these three behaviors, dMT mice exhibited comparable performance to wild-type controls (p>0.05 for all of the following parameters: distance traveled: 17.76±0.91 m; stereotypy counts: 1756±102; center time: 108.8±14.6 sec).

FIG. 12. Trace fear conditioning task. Mice of different genotypes (WT, n=15 mice; dnPAK TG, n=12 mice; FMR1 KO, n=15 mice; dMT, n=9 mice) were subjected to the trace fear conditioning task according to standard procedures. On day 1 (“conditioning”), mice were placed into a training chamber for 60 seconds before the onset of a 15-second white noise tone. Another 30 seconds later, mice received a 1-second shock (0.7 mA intensity). Thus, one trial is composed of tone, 30 seconds blank time (also called “trace”), and then shock. Seven trials with an intertrial interval (ITI) of 210 seconds were performed. To examine whether mice remember this association, on day 2 (“tone test”), mice were placed into a new chamber with a different shape and smell from the first chamber. After 60 seconds, a 15-second tone was repeated for seven times with an ITI of 210 seconds. Video images were digitized and the percentage of freezing time during each ITI was analyzed by Image FZ program. Freezing was defined as the absence of all but respiratory movement for a 1-second period. On day 1 (“Conditioning”), the four genotypes of mice exhibited comparable amounts of freezing pre-conditioning (“Baseline”) and post-conditioning in all trials. At the 24-hour tone test, the four genotypes exhibit comparable amounts of pre-tone freezing (ANOVA p>0.05). However, for tone-dependent freezing, FMR1 KO mice and dnPAK TG mice exhibited a significant reduction compared to wild-type controls (ANOVA for each tone session, p<0.05; for FMR1 KO versus WT, p<0.05 for session 1 and p<0.01 for sessions 2 to 7; for dnPAK TG versus WT, p>0.05 for session 1 and p<0.01 for sessions 2 to 7). dMT mice also showed freezing deficits during the first several tone sessions (sessions 1 to 4) compared to wild-type controls (p<0.05). However, with additional tone sessions (sessions 5 to 7), freezing by dMT mice caught up to that of wild-type controls (p>0.05). “n.s.”: not statistically different. *:p<0.05; **:p<0.01; ***: p<0.001.

FIG. 13. Quantification of mean tone-induced freezing. Average freezing for tone sessions 1 to 4: ANOVA p<0.05. dMT mice showed freezing deficits compared to wild-type controls (p<0.05), but the deficits in dMT mice were less pronounced compared to dnPAKTG (p<0.01) or FMR1 KO mice (p<0.01). Average freezing for tone sessions 5 to 7: ANOVA p<0.05. Freezing level in dMT mice was not significantly different from wild-type controls (p>0.05) and there were trends in its difference from dnPAK TG (p=0.12) or FMR1 KO mice (p=0.07).

FIG. 14. Immunoprecipitation. Immunoprecipitations were performed using either rabbit serum (negative control), α-PAK1, or α-PAK1 plus a blocking peptide. Western blots were probed for either PAK1 or FMRP. α-PAK1, but not rabbit serum, immunoprecipitates FMRP in this assay. The specificity of the interaction was tested by including a blocking peptide specific for α-PAK1 in the immunoprecipitation reaction. Input: 2% of the extract used for a single immunoprecipitation was loaded on the gel.

FIG. 15. GST pull down. FMRP was produced by in vitro-translation. GST and GST-tagged PAK1 were purified from a bacterial expression system. Wild type FMRP and the FMRP mutants depicted in FIG. 17 were used in the GST-pull down assay. “Input”: in vitro translated FMRP sample before the reaction was carried out; “MW”: molecular weight standard.

FIG. 16. Characterization of the interaction between PAK1 and various FMRP variants in vitro. In vitro-translated FMRP variants were incubated with GST or GST-PAK1 and glutathione sepharose beads. The complexes isolated by this method were subjected to SDS-PAGE and Western blotted for FMRP. “Input”: 10% of in vitro-translated FMRP sample before the binding reaction was carried out was loaded on the gel.

FIG. 17. Wild type and mutant FMRP domain structure. FIG. 17 (adapted from Mazroui et al., 2003, Hum. Mol. Genet. 12:3087; incorporated herein by reference) shows a schematic structure of FMRP, highlighting various functional domains including three RNA-binding motifs (RGG, KH1, and KH2) and the phosphorylation site (S499, represented by a white asterisk). The constructs used for in vitro binding included full length (WT), truncated (ΔRGG, ΔS499 and ΔKH), or mutated (1304N) FMRP. ΔRGG refers to the FMRP variant with a deletion of the RGG box at amino acids 526-555. The deleted area in ΔS499 spans amino acids 443-527 and includes the phosphorylation site, S499_{;} as well as putative phosphorylation sites. The isoleucine to asparagine missense mutation in the KH2 domain mimics that previously reported in a human FXS patient (1304N, represented by a black asterisk). The ΔKH deletion mutant lacks both KH domains in tandem corresponding to amino acids 207-425. Adapted from Mazroui et al., 2003, Hum. Mol. Genet., 12:3087; incorporated herein by reference; numbering refers to the amino acid positions designated by the SwissProt Q06787 entry.

### DEFINITIONS

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. In some embodiments, amino acids, including carboxy- and/or amino-terminal amino acids in peptides, are modified by methylation, amidation, acetylation, and/or substitution with other chemical groups that can change the peptide's circulating half-life without adversely affecting their activity. In some embodiments, amino acids participate in a disulfide bond. The term “amino acid” is used interchangeably with “amino acid residue,” and, in some embodiments, refers to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal is a transgenic animal, genetically-engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Characteristic portion: As used herein, the term a “characteristic portion” of a substance, in the broadest sense, is one that shares some degree of sequence and/or structural identity and/or at least one functional characteristic with the relevant intact substance. For example, a “characteristic portion” of a small molecule is one that shares at least one functional characteristic with the relevant intact substance. In some embodiments, a characteristic portion is biologically active.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; (4) post-translational modification of a polypeptide or protein.

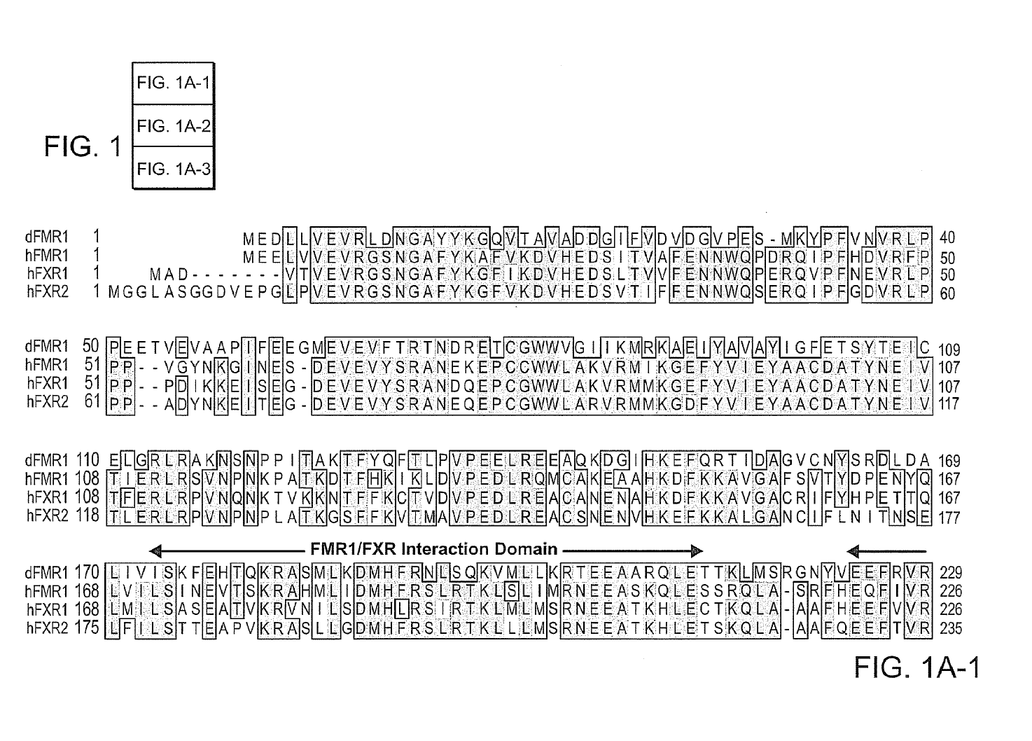

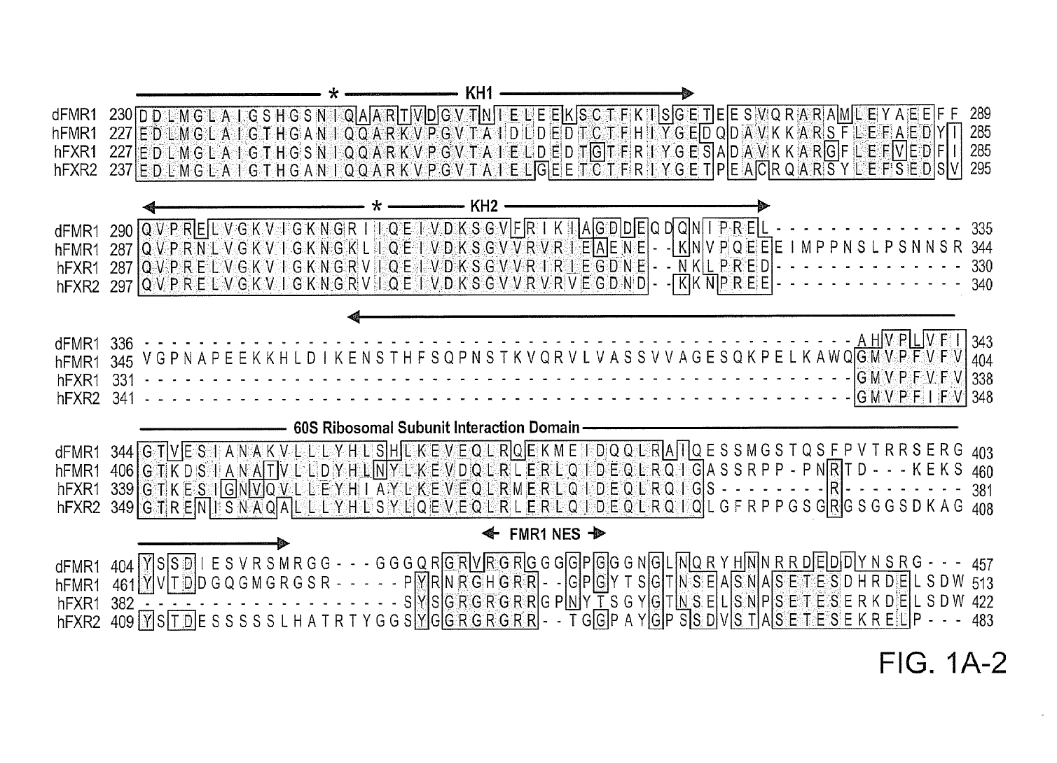

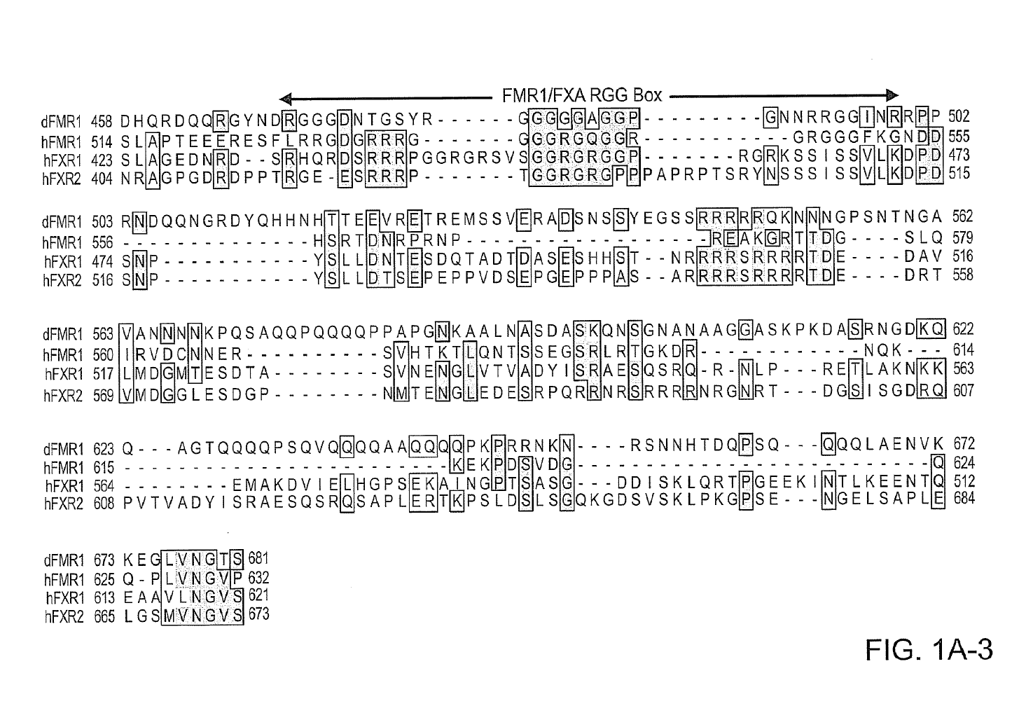

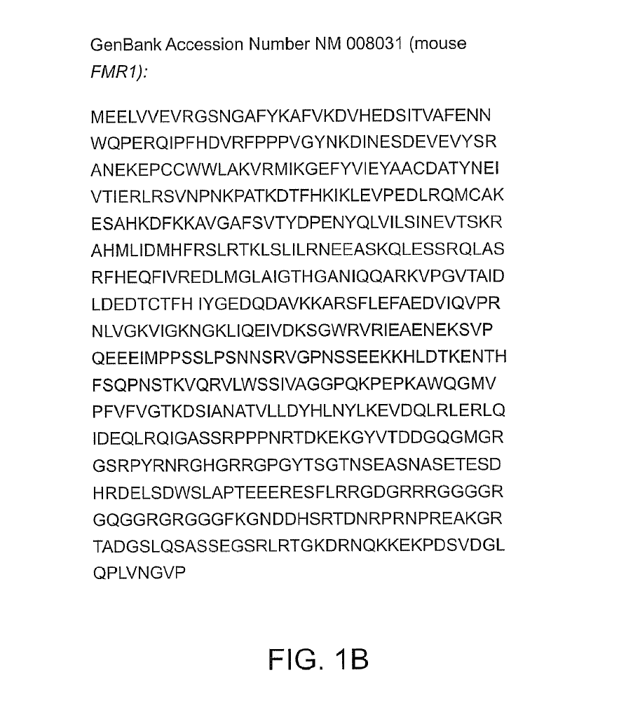

FMR1: As used herein, the “FMR1” gene refers to any nucleotide sequence that encodes the fragile X mental retardation protein (FMRP) and/or a characteristic portion thereof. Representative examples of FMR1 nucleotide sequences depicted in FIG. 1 include GenBank Accession Number AF305881 and GenBank Accession Number L29074. The “FXR” gene is homologous to FMR1 and may compensate for loss of FMR1 function. Representative examples of FXR nucleotide sequences include, but are not limited to, FXR1 (GenBank Accession Number U25165) and FXR2 (GenBank Accession Number U31501). FXR1 and FXR2 genes depicted in FIG. 1 are homologous to FMR1 and may compensate for loss of FMR1 function. In some embodiments, an FMR1 gene comprises any nucleotide sequence that shares about 70%, about 80%, about 90%, about 95%, or greater than 95% sequence identity with the sequences of GenBank Accession Numbers AF305881, L29074, U25165, and/or U31501.

Fragile X mental retardation protein (FMRP): As used herein, “fragile X mental retardation protein,” or “FMRP,” refers to any protein product of the fragile X mental retardation (FMR1) nucleotide sequence, and/or a characteristic portion thereof. Representative examples of FMRP amino acid sequences depicted in FIG. 1 include, but are not limited to, GenBank Accession Number AAG22045 and GenBank Accession Number AAB 18829. The “FXR” protein is homologous to FMRP and may compensate for loss of FMRP function. Representative examples of FXR amino acid sequences include, but are not limited to, FXR1 (GenBank Accession Number AAC50155) and FXR2 (GenBank Accession Number AAC50292). In some embodiments, FMRP comprises any amino acid sequence that shares about 70%, about 80%, about 90%, about 95%, or greater than 95% sequence identity with the sequences of GenBank Accession Numbers AAG22045, AAB18829, AAC50155, and/or AAC50292.

Fragile X Syndrome (FXS): As used herein, in some embodiments “fragile X syndrome,” or “FXS,” refers to a disease, disorder, and/or condition characterized by one or more of the following symptoms: (1) behavioral symptoms, including but not limited to hyperactivity, stereotypy, anxiety, seizure, impaired social behavior, and/or cognitive delay; (2) defective synaptic morphology, such as an abnormal number, length, and/or width of dendritic spines; and/or (3) defective synaptic function, such as enhanced long-term depression (LTD) and/or reduced long-term potentiation (LTP). In some embodiments, FXS refers to a disease, disorder, and/or condition caused by and/or associated with one or more of the following: (1) a mutation in FMR1 (the nucleotide sequence encoding FMRP); (2) defective FMR1 expression; (3) increased and/or decreased expression of FMRP; (4) defective FMRP function; (5) increased and/or decreased expression of FMRP's natural binding partners; (6) an increased and/or decreased ability of FMRP to bind to its natural binding partners; (7) decreased or absent arginine methylation of FMRP; (8) the increased methylation of FMR1 CpG repeats in the 5′ UTR of exon 1; (9) the mislocalization or misexpression of FMRP within the cell or within the organism; (10) the inhibition of function of FMR1 transcription factors Sp1 and/or NRF1; (11) an increased and/or decreased ability of FMRP to associate with polysomes; (12) the loss of function of FXR1 and/or FXR2, which are homologous to FMR1 and may compensate for loss of FMR1 function; (13) the decreased ability of FMRP to recognize RNA secondary structures that FMRP normally recognizes (such as intramolecular G quartet and/or FMRP “kissing complex”); (14) an increased and/or decreased ability of FMRP to interact with miRNAs and/or members of the miRNA pathway; (15) an increased and/or decreased ability of FMRP to interact with its known target RNAs, such as RNAs encoding Racl, microtubule-associated protein 1B, activity-regulated cytoskeleton-associated protein, and/or alpha-calcium/calmodulin-dependent protein kinase II; (16) an increased and/or decreased ability of phosphatase PP2A to act on FMRP (PP2A is thought to bring about translational repression activity of FMRP); (17) the increased and/or decreased activity of mGluR5, which is known to decrease activity of phosphatase PP2A (18) exaggerated signaling in mGluR pathways (Bear et al., 2004, Trends Neurosci., 27:370; incorporated herein by reference); (19) disruption of a KH domain of FMRP, which decreases the ability of FMRP to interact with PAK; and/or (20) an 1304N mutation in FMRP, which decreases the ability of FMRP to interact with PAK. The teachings of the present invention described herein with respect to FXS are applicable to other neurodevelopmental disorders including, for example, premature ovarian failure (POF), fragile X-associated tremor ataxia (FXTAS), and/or other neurodevelopmental disorders, including, but not limited to, various forms of mental retardation and/or autism spectrum disorders (ASD). Furthermore, the teachings of the present invention described herein with respect to FXS are applicable to any disease, disorder, and/or condition caused by and/or associated with one or more of the following: (1) a mutation in FMR1 (the nucleotide sequence encoding FMRP); (2) defective FMR1 expression; (3) increased and/or decreased expression of FMRP; (4) defective FMRP function; (5) increased and/or decreased expression of FMRP's natural binding partners; (6) an increased and/or decreased ability of FMRP to bind to its natural binding partners; (7) decreased or absent arginine methylation of FMRP; (8) the increased methylation of FMR1 CpG repeats in the 5′ UTR of exon 1; (9) the mislocalization or misexpression of FMRP within the cell or within the organism; (10) the inhibition of function of FMR1 transcription factors Sp1 and/or NRF1; (11) an increased and/or decreased ability of FMRP to associate with polysomes; (12) the loss of function of FXR1 and/or FXR2, which are homologous to FMR1 and may compensate for loss of FMR1 function; (13) the decreased ability of FMRP to recognize RNA secondary structures that FMRP normally recognizes (such as intramolecular G quartet and/or FMRP “kissing complex”); (14) an increased and/or decreased ability of FMRP to interact with miRNAs and/or members of the miRNA pathway; (15) an increased and/or decreased ability of FMRP to interact with its known target RNAs, such as RNAs encoding Rac1, microtubule-associated protein 1B, activity-regulated cytoskeleton-associated protein, and/or alpha-calcium/calmodulin-dependent protein kinase II; (16) an increased and/or decreased ability of phosphatase PP2A to act on FMRP (PP2A is thought to bring about translational repression activity of FMRP); (17) the increased and/or decreased activity of mGluR5, which is known to decrease activity of phosphatase PP2A; (18) exaggerated signaling in mGluR pathways (Bear et al., 2004, Trends Neurosci., 27:370; incorporated herein by reference); (19) disruption of a KH domain of FMRP, which decreases the ability of FMRP to interact with PAK; and/or (20) an 1304N mutation in FMRP, which decreases the ability of FMRP to interact with PAK.

Functional: As used herein, a “functional” biological molecule is a biological molecule in a form in which it exhibits a property and/or activity by which it is characterized.

Gene: As used herein, the term “gene” has its meaning as understood in the art. The term “gene” includes, in some embodiments, gene regulatory sequences (e.g., promoters, enhancers, etc.) and/or intron sequences. It will further be appreciated that definitions of gene include references to nucleic acids that do not encode proteins but rather encode functional RNA molecules such as tRNAs, RNAi-inducing agents, etc. For the purpose of clarity we note that, as used in the present application, the term “gene” generally refers to a portion of a nucleic acid that encodes a protein; the term optionally encompasses regulatory sequences. This definition is not intended to exclude application of the term “gene” to non-protein-coding expression units but rather to clarify that, in most cases, the term as used in this document refers to a protein-coding nucleic acid.

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g. between nucleic acid molecules (e.g. DNA molecules and/or RNA molecules) and/or between polypeptide molecules. Calculation of the percent identity of two nucleic acid sequences, for example, is performed by aligning the two sequences for optimal comparison purposes (e.g., gaps are introduced in one or both of a first and a second nucleic acid sequences for optimal alignment and non-identical sequences are disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences is determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences is, alternatively, determined using the GAP program in the GCG software package using an NWSgapdna.CMP matrix.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. In some embodiments, isolated substances and/or entities are separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Natural binding partner: As used herein, the term “natural binding partner” refers to any substance that binds to PAK and/or FMRP. In some embodiments, the substance binds directly, and in some embodiments, the substance binds indirectly. In some embodiments, a natural binding partner substance includes a protein, nucleic acid, lipid, carbohydrate, glycoprotein, proteoglycan, and/or small molecule that binds to either PAK and/or FMRP. In some embodiments, a change in the interaction between PAK and/or FMRP and a natural binding partner manifests itself as an increased and/or decreased probability that the interaction forms. In some embodiments, a change in the interaction between PAK and/or FMRP and a natural binding partner manifests itself as an increased and/or decreased concentration of PAK and/or FMRP/natural binding partner complex within the cell. This can result in an increased and/or decreased activity of PAK and/or FMRP. The present invention identifies FMRP as a novel natural binding partner of PAK. Other natural binding partners of PAK include PAK substrates and/or other substances that interact with PAK. Examples of natural binding partners of PAK include; but are not limited to; Myosin light chain kinase (MLCK); regulatory Myosin light chain (R-MLC); Myosin I heavy chain; Myosin II heavy chain; Myosin VI; Caldesmon; Desmin; Op18/stathmin; Merlin; Filamin A; LIM kinase (LIMK); Ras; Raf; Mek; p47^{phox}; BAD; caspase 3; estrogen and/or progesterone receptors; RhoGEF; GEF-H1; NET1; Gαz; phosphoglycerate mutase-B; RhoGDI; prolactin; p41^{Arc}; Aurora-A; Rac/Cdc42; CIB; sphingolipids; G-protein β and/or γ subunits; PIX/COOL; GIT/PKL; Paxillin; Nef; NESH; SH3-containing proteins (e.g. Nck and/or Grb2); kinases (e.g. Akt, PDK1, PI 3-kinase/p85, Cdk5, Cdc2, Src kinases, Abl, and/or protein kinase A (PKA)); and/or phosphatases (e.g. phosphatase PP2A, POPX1, and/or POPX2) (Bokoch et al., 2003, Annu. Rev. Biochem., 72:743; and Hofmann et al., 2004, J. Cell Sci., 117:4343; both of which are incorporated herein by reference). Prominent upstream effectors of PAK include, but are not limited to, PDK1, PDK2, PI3K, and/or NMDARs.

PAK activity: As used herein, the term “PAK activity” refers to any activity and/or function of p21-activated kinase. Examples of “PAK activity” include, but are not limited to, PAK binding to other substances, PAK kinase activity, autophosphorylation, translocation, etc. “PAK activity” is used interchangeably with “PAK function.”

PAK Inhibitor: As used herein, the term “PAK inhibitor” refers to any substance that directly and/or indirectly decreases the activity and/or levels of PAK. In some embodiments, PAK inhibitors inhibit, decrease, and/or abolish the level of PAK mRNA and/or protein; an activity of PAK; the half-life of PAK mRNA and/or protein; and/or the interaction between PAK and its natural binding partners (e.g., a substrate for a PAK kinase, a Rac protein, a cdc42 protein, and/or FMRP), as measured using standard methods. In some embodiments, mRNA expression levels are determined using RNase protection assays and/or in situ hybridization assays, and/or the level of protein are determined using Western and/or immunohistochemistry analysis. In some embodiments, phosphorylation levels of signal transduction proteins downstream of PAK activity are measured using standard assays. In some embodiments, PAK inhibitors reduce, abolish, and/or remove the binding between PAK and FMRP. Thus, binding between PAK and FMRP is stronger in the absence of the inhibitor than in its presence. Put another way, a PAK inhibitor increases the Kₘ of binding between PAK and FMRP. Alternatively or additionally, PAK inhibitors inhibit the kinase activity of PAK. In some embodiments, PAK inhibitors inhibit the ability of PAK to phosphorylate FMRP. PAK inhibitors include inorganic and/or organic compounds. In accordance with the present invention, PAK inhibitors are small molecules.

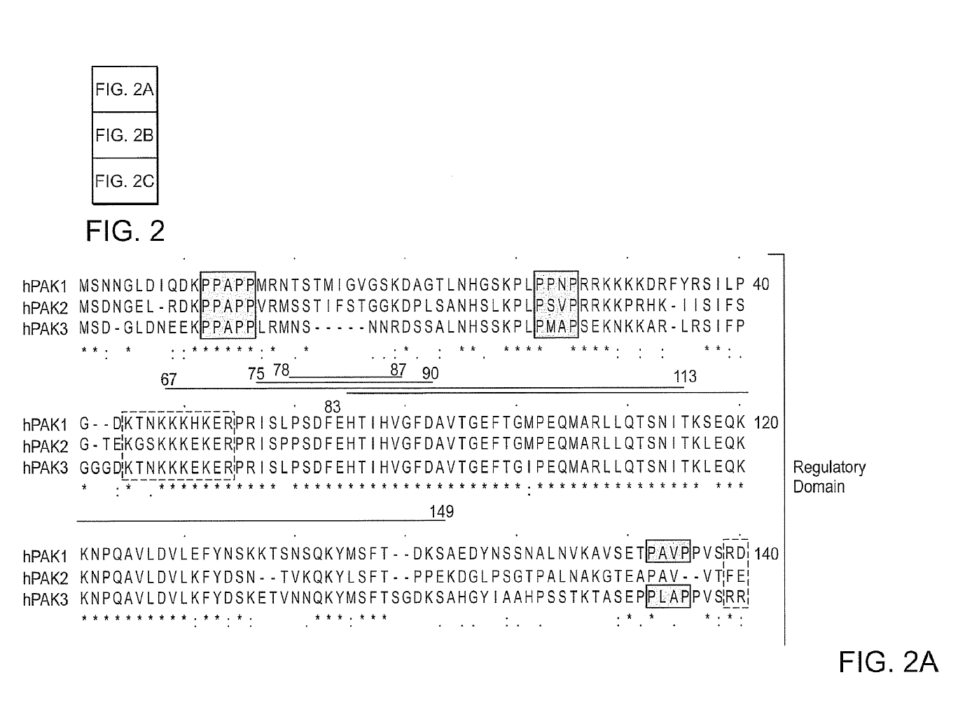

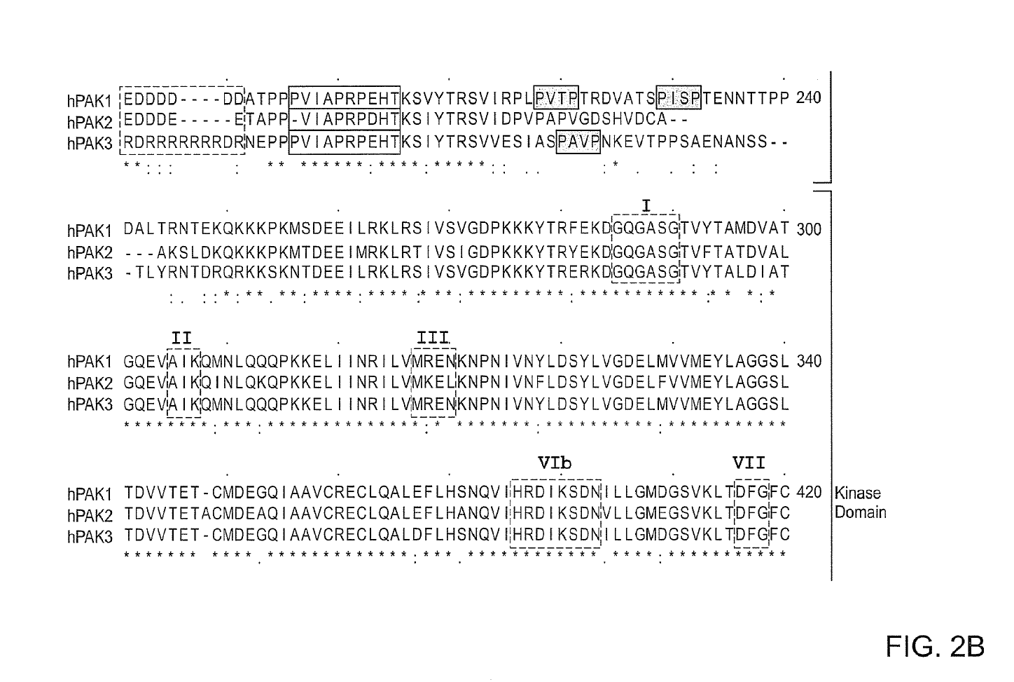

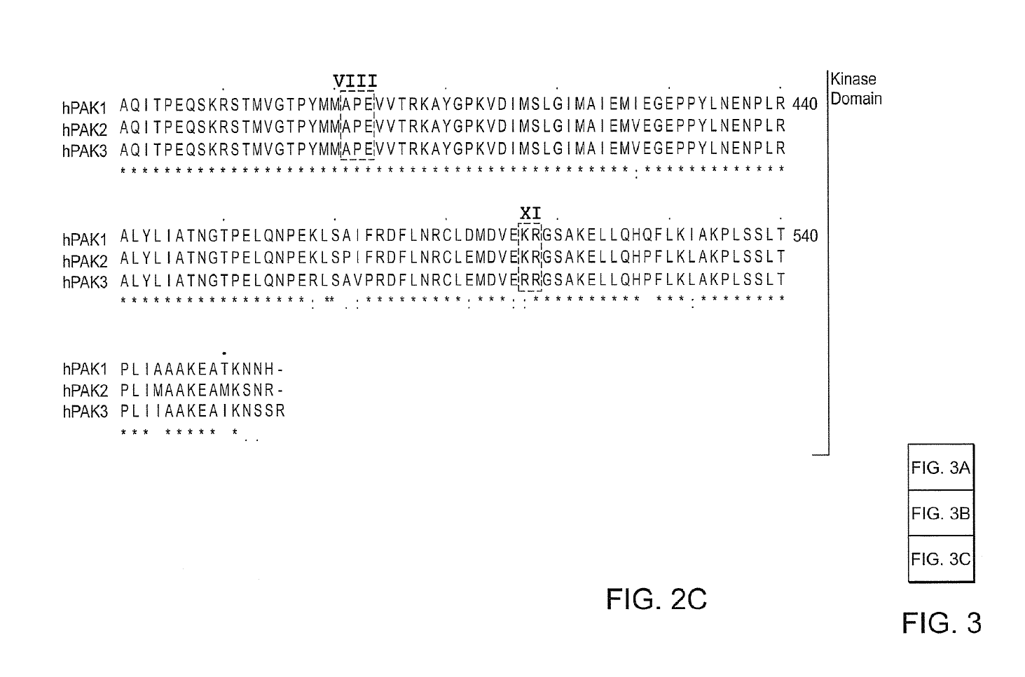

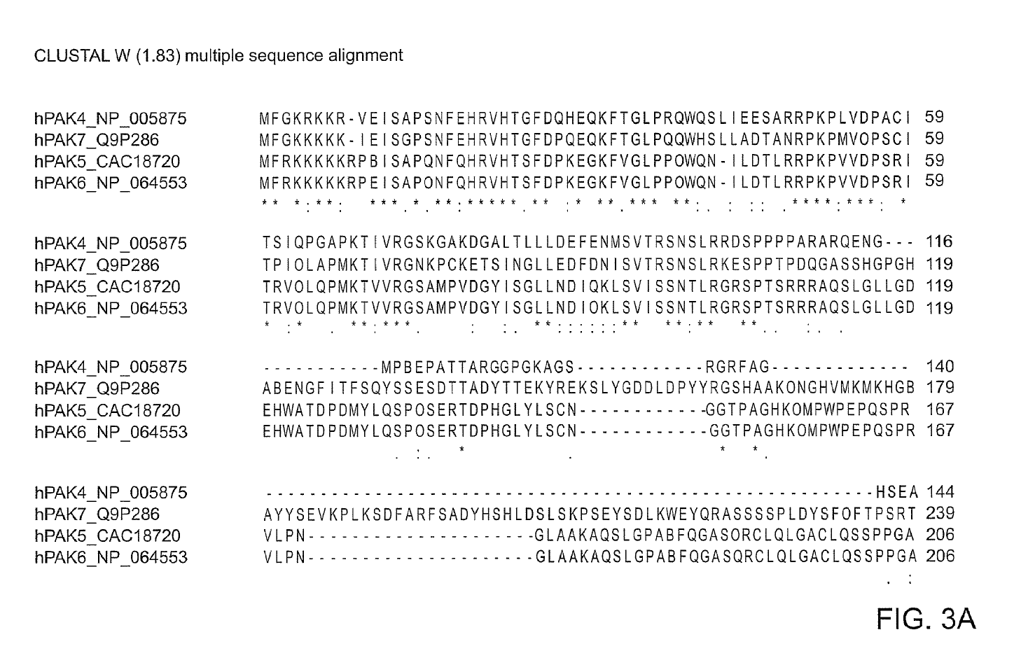

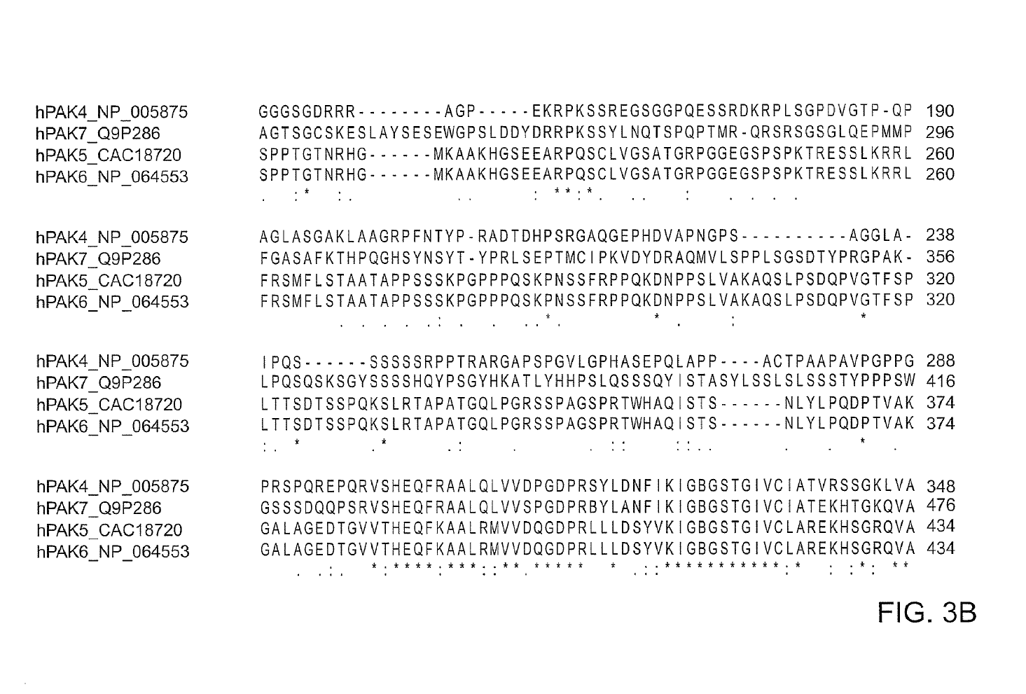

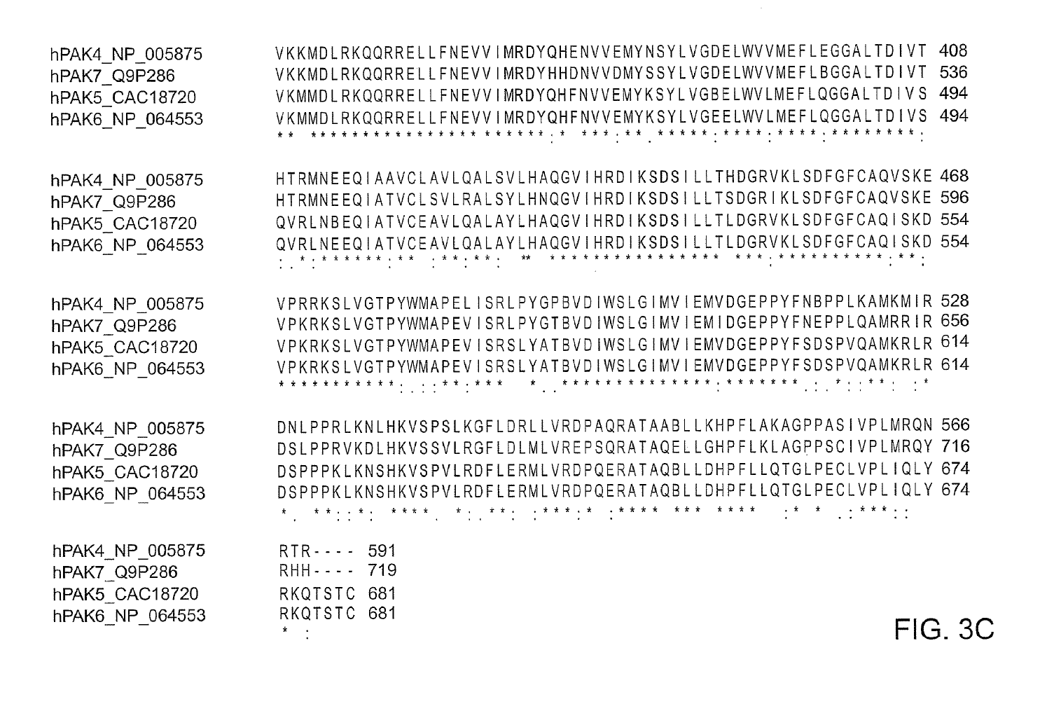

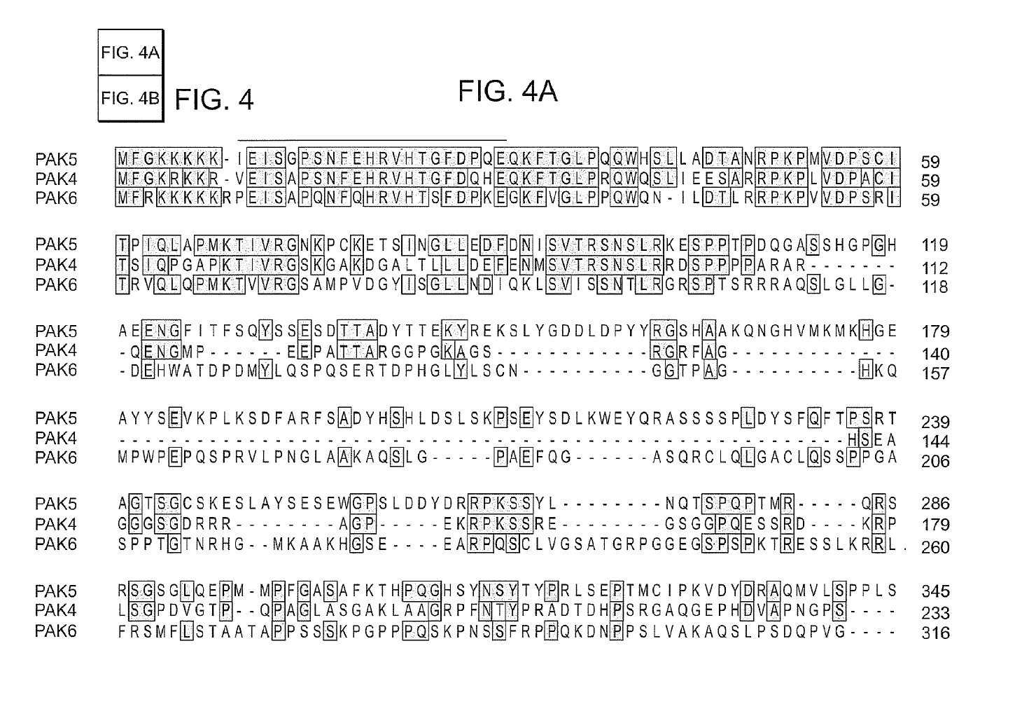

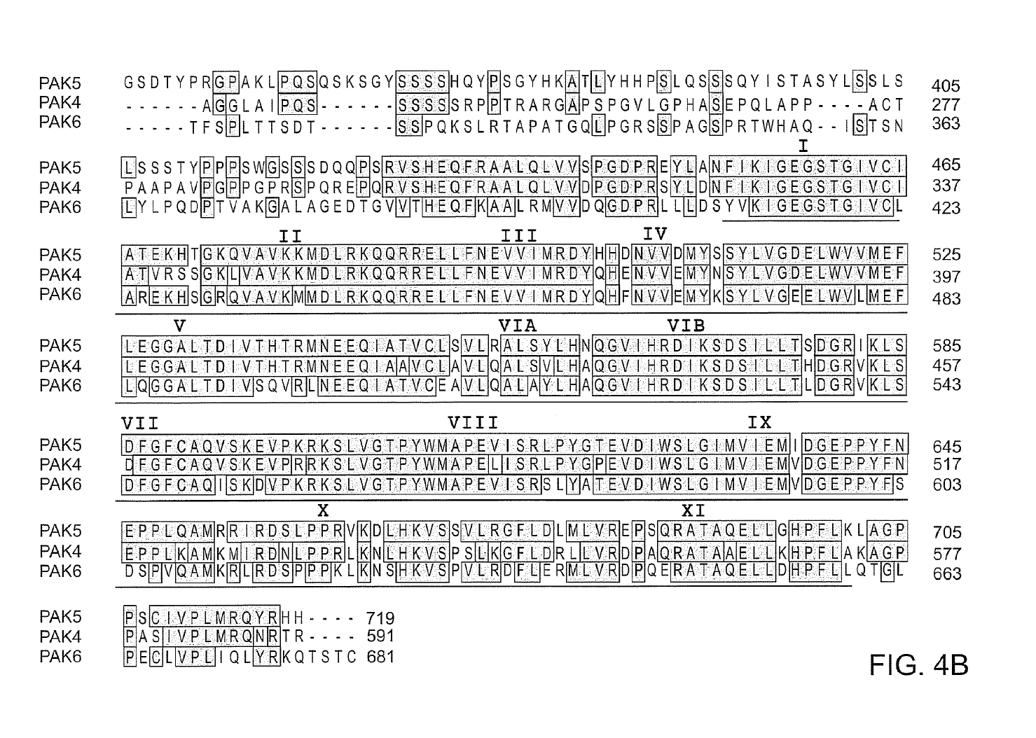

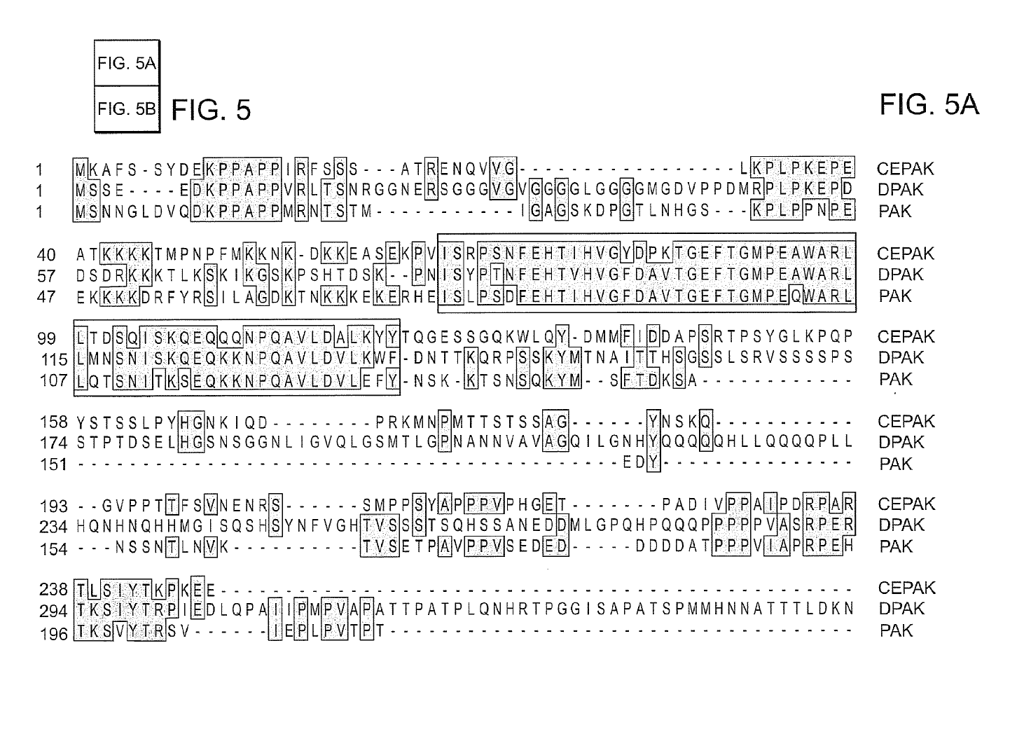

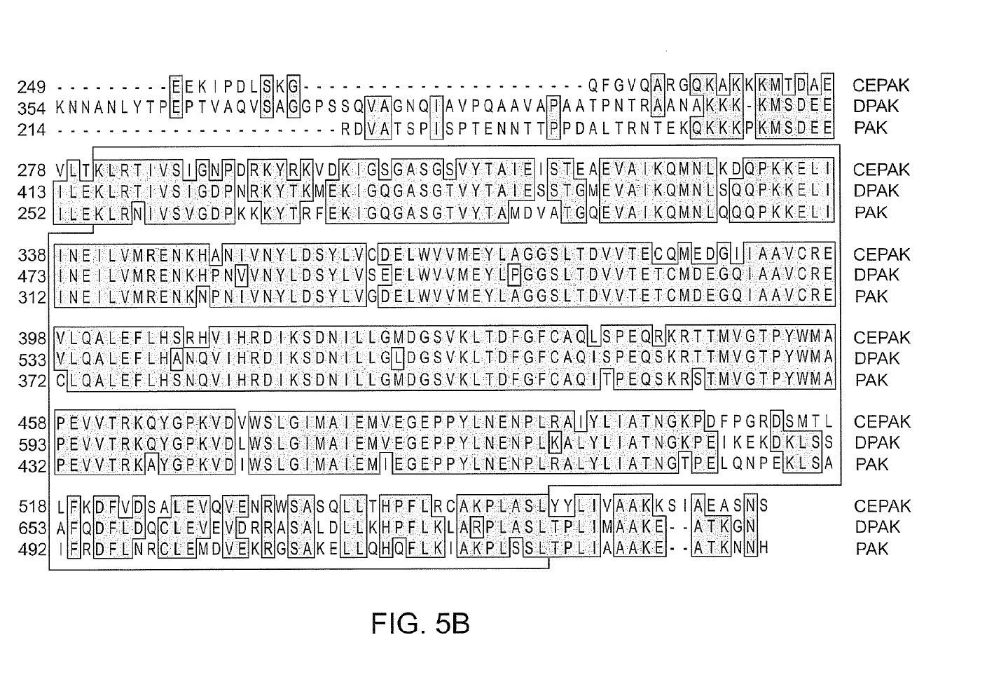

PAK protein: As used herein the term “PAK protein” refers to a protein that belongs in the family of PAK serine/threonine protein kinases. These include mammalian isoform identified, e.g., PAK1, PAK2, PAK3, PAK4, PAK5, and/or PAK6; and/ or lower eukaryotic isoforms, such as the yeast Ste20 (Leberter et al., 1992, EMBO J, 11:4805; incorporated herein by reference) and/or the Dictyostelium single-headed myosin I heavy chain kinases (Wu et al., 1996, J. Biol. Chem., 271:31787; incorporated herein by reference). Representative examples of PAK amino acid sequences depicted in FIGS. 2, 3, 4, and 5 include, but are not limited to, human PAK1 (GenBank Accession Number AAA65441), human PAK2 (GenBank Accession Number AAA65442), human PAK3 (GenBank Accession Number AAC36097), human PAK 4 (GenBank Accession Numbers NP_{—}005875 and CAA09820), human PAK5 (GenBank Accession Numbers CAC18720 and BAA94194), human PAK6 (GenBank Accession Numbers NP_{—}064553 and AAF82800), human PAK7 (GenBank Accession Number Q9P286), C. elegans PAK (GenBank Accession Number BAA11844), D. melanogaster PAK (GenBank Accession Number AAC47094), and rat PAK1 (GenBank Accession Number AAB95646). Representative examples of PAK genes encoding PAK proteins include, but are not limited to, human PAK1 (GenBank Accession Number U24152), human PAK2 (GenBank Accession Number U24153), human PAK3 (GenBank Accession Number AF068864), human PAK4 (GenBank Accession Number AJ011855), human PAK5 (GenBank Accession Number AB040812), and human PAK6 (GenBank Accession Number AF276893). In some embodiments, a PAK protein comprises any amino acid sequence that shares about 70%, about 80%, about 90%, about 95%, or greater than 95% sequence identity with the sequences of GenBank Accession Numbers AAA65441, AAA65442, AAC36097, NP_{—}005875, CAA09820, CAC18720, BAA94194, NP_{—}064553, AAF82800, Q9P286, BAA11844, AAC47094, and/or AAB95646. In some embodiments, a PAK gene comprises any nucleotide sequence that shares about 70%, about 80%, about 90%, about 95%, or greater than 95% sequence identity with the sequences of GenBank Accession Numbers U24152, U24153, AF068864, AJ011855, AB040812, and/or AF276893.

Protein: As used herein, the term “protein” refers to a polypeptide (i.e., a string of at least two amino acids linked to one another by peptide bonds). In some embodiments, proteins include moieties other than amino acids (e.g., glycoproteins, proteoglycans, etc.) and/or otherwise processed or modified. A “protein” includes a complete polypeptide chain as produced by a cell (with or without a signal sequence), or a characteristic portion thereof. A protein sometimes includes more than one polypeptide chain, for example linked by one or more disulfide bonds or associated by other means. In some embodiments, Polypeptides contain L-amino acids, D-amino acids, or both and contain any of a variety of amino acid modifications or analogs. Useful modifications include, e.g., terminal acetylation, amidation, etc. In some embodiments, proteins comprise natural amino acids, non-natural amino acids, synthetic amino acids, and combinations thereof. The term “peptide” is generally used to refer to a polypeptide having a length of less than about 100 amino acids.

Small Molecule: In general, a “small molecule” is an organic molecule that is less than about 5 kilodaltons (Kd) in size. In some embodiments, the small molecule is less than about 4 Kd, 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. Typically, in accordance with the present invention, small molecules are not proteins, polypeptides, oligopeptides, peptides, polynucleotides, oligonucleotides, polysaccharides, glycoproteins, proteoglycans, etc. A derivative of a small molecule refers to a molecule that shares the same structural core as the original small molecule, but which can be prepared by a series of chemical reactions from the original small molecule. As one example, a pro-drug of a small molecule is a derivative of that small molecule. An analog of a small molecule refers to a molecule that shares the same or similar structural core as the original small molecule, and which is synthesized by a similar or related route, or art-recognized variation, as the original small molecule.

Subject: As used herein, the term “subject” or “patient” refers to any organism to which a composition of this invention may be administered, e.g., for experimental, diagnostic, prophylactic, and/or therapeutic purposes. Typical subjects include animals (e.g., mammals such as mice, rats, rabbits, non-human primates, and humans; insects; worms; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” FXS and/or other neurodevelopmental disorders has been diagnosed with or displays one or more symptoms of FXS and/or other neurodevelopmental disorders.

Susceptible to: An individual who is “susceptible to” FXS has not been diagnosed with FXS and/or may not exhibit symptoms of FXS but is characterized by one or more of the following: (1) a mutation in FMR1 (the nucleotide sequence encoding FMRP); (2) defective FMR1 expression; (3) increased and/or decreased expression of FMRP; (4) defective FMRP function; (5) increased and/or decreased expression of FMRP's natural binding partners; (6) an increased and/or decreased ability of FMRP to bind to its natural binding partners; (7) decreased or absent arginine methylation of FMRP; (8) the increased methylation of FMR1 CpG repeats in the 5′ UTR of exon 1; (9) the mislocalization or misexpression of FMRP within the cell or within the organism; (10) the inhibition of function of FMR1 transcription factors Sp1 and/or NRF1; (11) an increased and/or decreased ability of FMRP to associate with polysomes; (12) the loss of function of FXR1 and/or FXR2, which are homologous to FMR1 and may compensate for loss of FMR1 function; (13) the decreased ability of FMRP to recognize RNA secondary structures that FMRP normally recognizes (such as intramolecular G quartet and/or FMRP “kissing complex”); (14) an increased and/or decreased ability of FMRP to interact with miRNAs and/or members of the miRNA pathway; (15) an increased and/or decreased ability of FMRP to interact with its known target RNAs, such as RNAs encoding Rac1, microtubule-associated protein 1B, activity-regulated cytoskeleton-associated protein, and/or alpha-calcium/calmodulin-dependent protein kinase II; (16) an increased and/or decreased ability of phosphatase PP2A to act on FMRP (PP2A is thought to bring about translational repression activity of FMRP); (17) the increased and/or decreased activity of mGluR5, which is known to decrease activity of phosphatase PP2A; (18) exaggerated signaling in mGluR pathways (Bear et al., 2004, Trends Neurosci., 27:370; incorporated herein by reference); (19) disruption of a KH domain of FMRP, which decreases the ability of FMRP to interact with PAK; and/or (20) an 1304N mutation in FMRP, which decreases the ability of FMRP to interact with PAK. In some embodiments, an individual who is susceptible to FXS will develop FXS and/or other neurodevelopmental disorder. In some embodiments, an individual who is susceptible to FXS will not develop FXS and/or other neurodevelopmental disorder.

Therapeutically effective amount: As used herein, the term “therapeutically effective amount” means an amount of inventive PAK inhibitor that is sufficient, when administered to a subject suffering from or susceptible to FXS and/or other neurodevelopmental disorders, to treat, diagnose, prevent, and/or delay the onset of the FXS and/or other neurodevelopmental disorders symptom(s) and/or condition(s).

Therapeutic agent: As used herein, the phrase “therapeutic agent” refers to any agent that, when administered to a subject, has a therapeutic effect and/or elicits a desired biological and/or pharmacological effect.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition (e.g., FXS and/or other neurodevelopmental disorders). In one embodiment, treatment is administered to a subject who does not exhibit signs of a disease and/or exhibits only early signs of the disease for the purpose of decreasing the risk of developing pathology associated with the disease.

### DETAILED DESCRIPTION OF CERTAIN PREFERRED EMBODIMENTS OF THE INVENTION

In some embodiments, the present invention provides methods for inhibiting PAK with small molecule PAK inhibitors. In some embodiments, the present invention provides methods for treating fragile X syndrome (FXS) and/or other neurodevelopmental disorders comprising administering one or more small molecule PAK inhibitors to a subject susceptible to, suffering from, and/or exhibiting symptoms of treat FXS and/or other neurodevelopmental disorder. In accordance with some embodiments, compositions are provided comprising at least one small molecule PAK inhibitor and a pharmaceutically acceptable excipient.

p21-Activated Kinase (PAK)

PAK, a family of serine-threonine kinases that is composed of at least three members, PAK1, PAK2 and/or PAK3, functions downstream of the small GTPases Rac and/or Cdc42 to regulate multiple cellular functions, including motility, morphogenesis, angiogenesis, and/or apoptosis (Bokoch et al., 2003, Annu. Rev. Biochem., 72:743; and Hofmann et al., 2004, J. Cell Sci., 117:4343; both of which are incorporated herein by reference). GTP-bound Rac and/or Cdc42 bind to inactive PAK, releasing steric constraints imposed by a PAK autoinhibitory domain and/or permitting PAK auto-phosphorylation and/or activation. Numerous autophosphorylation sites have been identified that serve as markers for activated PAK.

Prominent downstream targets of mammalian PAK include, but are not limited to, substrates of PAK kinase, such as Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47^{phox}, BAD, caspase 3, estrogen and/or progesterone receptors, RhoGEF, GEF-H1, NET1, Gαz, phosphoglycerate mutase-B, RhoGDI, prolactin, p41^{Arc}, and/or Aurora-A (Bokoch et al., 2003, Annu. Rev. Biochem., 72:743; and Hofmann et al., 2004, J. Cell Sci., 117:4343; both of which are incorporated herein by reference). Other substances known to bind to PAK in cells include CIB; sphingolipids; G-protein β and/or γ subunits; PIX/COOL; GIT/PKL; Nef; Paxillin; NESH; SH3-containing proteins (e.g. Nck and/or Grb2); kinases (e.g. Akt, PDK1, PI 3-kinase/p85, Cdk5, Cdc2, Src kinases, Abl, and/or protein kinase A (PKA)); and/or phosphatases (e.g. phosphatase PP2A, POPX1, and/or POPX2)

Prominent upstream effectors of PAK include, but are not limited to, PDK1, PDK2, PI3K, and/or NMDARs.

### Small Molecule PAK Inhibitors

PAK inhibitors have been described in the art as possible substances for use in the treatment of cancer, endometriosis, urogenital disorders, macropinocytosis, viral infection, vascular permeability, joint disease, lymphocyte activation, muscle contraction, and/or diabetes (see, for example, U.S. Pat. Nos. 5,863,532, 6,191,169, and 6,248,549; U.S. Patent Applications 2002/0045564, 2002/086390, 2002/106690, 2002/142325, 2003/124107, 2003/166623, 2004/091992, 2004/102623, 2004/208880, 2005/0203114, 2005/037965, 2005/080002, and 2005/233965; EP Patent Publication 1492871; Kumar et al., 2006, Nat. Rev. Cancer, 6:459; Eswaren et al., 2007, Structure, 15:201; all of which are incorporated herein by reference).

PAK inhibitors have also been described in the art as possible substances for use in the treatment of neurological disorders characterized by nerve damage and/or neurodegeneration. Such disorders include Parkinson's disease, Alzheimer's disease, prion-related diseases, neurofibromatosis, stroke-induced nerve damage, and/or diseases associated with nerve injury due to ischaemia and/or anoxia (see, for example, U.S. Pat. Nos. 5,952,217 and 6,046,224; U.S. Patent Application 2006/088897; and PCT applications WO 99/02701 and WO 04/07507; all of which are incorporated herein by reference).

However, PAK inhibitors have not previously been described in the art as possible substances for use in the treatment of neurodevelopmental disorders, including but not limited to FXS, premature ovarian formation (POF), fragile X-associated tremor ataxia (FXTAS), various forms of mental retardation, and/or autism spectrum disorders (ASD). The present invention encompasses the use of specific small molecule PAK inhibitors in the treatment of FXS, POF, FXTAS, and/or other neurodevelopmental disorders.

PAK inhibitors to be used in accordance with the present invention are small molecules. In general, a “small molecule” is an organic molecule that is less than about 5 kilodaltons (Kd) in size. In some embodiments, the small molecule is less than about 4 Kd, 3 Kd; about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. Typically, in accordance with the present invention, small molecules are not proteins, polypeptides, oligopeptides, peptides, polynucleotides, oligonucleotides, polysaccharides, glycoproteins, proteoglycans, etc. A derivative of a small molecule refers to a molecule that shares the same structural core as the original small molecule, but which can be prepared by a series of chemical reactions from the original small molecule. As one example, a pro-drug of a small molecule is a derivative of that small molecule. An analog of a small molecule refers to a molecule that shares the same or similar structural core as the original small molecule, and which is synthesized by a similar or related route, or art-recognized variation, as the original small molecule.

In various embodiments, a small molecule PAK inhibitor is a purified and/or unpurified synthetic organic molecule and/or naturally occurring organic molecule. In some embodiments, a small molecule PAK inhibitor is in solution and/or in suspension (e.g., in crystalline, colloidal, or other particulate form). In some embodiments, a small molecule PAK inhibitor is in the form of a monomer, dimer, oligomer, etc., and/or in a complex.

In some embodiments, small molecule PAK inhibitors are isolated from natural sources, such as animals, bacteria, fungi, plants, and/or marine samples. It will be understood that small molecule PAK inhibitors can be derived and/or synthesized from chemical compositions and/or man-made substances.

In some embodiments, small molecule PAK inhibitors target PAK directly. In some embodiments, small molecule PAK inhibitors target PAK indirectly. For example, PAK inhibitors might target downstream effectors of PAK, upstream effectors of PAK, and/or natural binding partners of PAK.

In some embodiments, exemplary small molecule PAK inhibitors that are used in accordance with the present invention include BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof.

In some embodiments, substances that inhibit serine/threonine kinase activity also inhibit PAK kinase activity.

In some embodiments, small molecule PAK inhibitors affect the ability of PAK to interact with its natural binding partners, including but not limited to FMRP. In certain embodiments, such binding blocks the interaction between PAK and its natural binding partners (for example, the interaction between PAK and FMRP). In some embodiments, such binding promotes the interaction between PAK and its natural binding partners. However, a small molecule PAK inhibitor need not necessarily bind directly to a catalytic and/or binding site, and binds, for example, to an adjacent site, such as an adjacent site in the PAK polypeptide. In some embodiments, a small molecule PAK inhibitor binds to another substance (for example, a protein, lipid, carbohydrate, etc. which is complexed with the enzyme), so long as its binding inhibits PAK activity.

In some embodiments, the FMRP KH domains facilitate FMRP's interaction with PAK. In specific embodiments, small molecule PAK inhibitors affect the integrity of one or more KH domains of FMRP and inhibit its ability to bind to PAK.

In some embodiments, a small molecule PAK inhibitor binds to a natural binding partner of PAK and inhibits and/or promotes the interaction of PAK with its natural binding partner. In some embodiments, a small molecule PAK inhibitor binds to PAK and inhibits and/or promotes the interaction of a natural binding partner of PAK with PAK. Some inhibitors that regulate PAK function in this manner have been described in the art. For example, U.S. Patent Application 2004/0208880 (incorporated herein by reference) describes compounds that inhibit PAK1 function by inhibiting its interaction with dynein light chain 1/protein inhibitors of nitric oxide synthase (DLC1/PIN). U.S. Patent Application 2004/0091907 (incorporated herein by reference) describes compounds that inhibit PAK4 by inhibiting its interaction with GEF/H1 (used to treat cancer). U.S. Patent Application 2002/0106690 (incorporated herein by reference) describes inhibitors that function by altering the interaction between PAK and the beta subunit of G-protein coupled receptors. U.S. Patent Application 2006/0088897 (incorporated herein by reference) describes substances that inhibit PAK by altering the interaction between PAK and SH3 domain-containing proteins.

In some embodiments, a small molecule PAK inhibitor bind to and/or compete for one or more sites on a relevant molecule, for example, a catalytic site and/or a binding site of PAK. In some embodiments, a small molecule PAK inhibitor interferes with and/or inhibits the binding of FMRP to PAK. In certain embodiments, a small molecule PAK inhibitor competes for an FMRP-binding region of PAK. In some embodiments, a small molecule PAK inhibitor competes for a PAK-binding region of FMRP.

Where small molecule inhibitors of an enzyme such as PAK are concerned, an inhibitor includes a small molecule isolated from a biological source (e.g. purified from tissues and/or cells) and/or by chemical synthesis and used to compete with the substrate for binding sites on the enzyme.

In some embodiments, small molecule PAK inhibitors are substances which bind to and/or block the kinase domain of PAK and/or the p21-binding domain of PAK, and/or the autophosphorylation sites of PAK.

In certain embodiments, small molecule PAK inhibitors function to alter the ability of PAK to phosphorylate its substrates.

In certain embodiments, small molecule PAK inhibitors function by altering the activity and/or expression of PAK activators. For example, U.S. Pat. No. 6,046,224 (incorporated herein by reference) describes agents that inhibit PAK function by blocking 12(S)HETE receptors. 12(S)HETE stimulates PAK activity, so blocking 12(S)HETE function indirectly inhibits PAK function.

In certain embodiments, small molecule PAK inhibitors comprise phosphatases that inhibit PAK function by removing phosphate groups from targets that are phosphorylated by PAK kinase.

Alternatively or additionally, small molecule PAK inhibitors affect PAK levels by increasing and/or decreasing transcription and/or translation of PAK, PAK substrates, and/or natural binding partners of PAK. In some embodiments, small molecule PAK inhibitors affect RNA and/or protein half-life, for example, by directly affecting mRNA and/or protein stability. In certain embodiments, small molecule PAK inhibitors cause the mRNA and/or protein to be more and/or less accessible and/or susceptible to nucleases, proteases, and/or the proteasome.

In some embodiments, small molecule PAK inhibitors affect the processing of mRNAs encoding PAK, PAK substrates, and/or natural binding partners of PAK. For example, small molecule PAK inhibitors function at the level of pre-mRNA splicing, 5′ end formation (e.g. capping), 3′ end processing (e.g. cleavage and/or polyadenylation), nuclear export, and/or association with the translational machinery and/or ribosomes in the cytoplasm.

In some embodiments, small molecule PAK inhibitors affect translational control and/or post-translational modification of PAK, PAK substrates, and/or natural binding partners of PAK. For example, small molecule PAK inhibitors function at the level of translation initiation, elongation, termination, and/or recycling. In some embodiments, small molecule PAK inhibitors function at the step of protein folding into secondary, tertiary, and/or quaternary structures. Alternatively or additionally, small molecule PAK inhibitors function at the level of intracellular transport (e.g. ER to Golgi transport, intra-Golgi transport, Golgi to plasma membrane transport, and/or secretion from the cell). In some embodiments, small molecule PAK inhibitors function at the level of post-translational modification (e.g. cleavage of signal sequences and/or the addition of entities such as methyl groups, phosphates, glycan moieties, etc.).

In some embodiments, small molecule PAK inhibitors cause the level of PAK mRNA and/or protein, an activity of PAK protein, the half-life of PAK mRNA and/or protein, the binding of PAK mRNA and/or protein to its natural binding partners, and/or the level and/or activity of a substance that phosphorylates a PAK kinase to decrease by at least about 5%, at least about 10%, at least about 20%, at least about 30%, at least about 40%, at least about 50%, at least about 60%, at least about 80%, at least about 90%, at least about 95%, or substantially 100%.

In some embodiments, small molecule PAK inhibitors are used alone and/or in conjunction with other substances which affect PAK activity.

### Pharmaceutical Compositions

The present invention provides specific small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) that are used, in some embodiments, in the treatment of FXS and/or other neurodevelopmental disorders. In some embodiments, the present invention provides pharmaceutical compositions comprising specific small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) and at least one pharmaceutically acceptable excipient. The present invention provides pharmaceutical compositions comprising a therapeutically effective amount of inventive small molecule PAK inhibitor(s) (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) appropriately formulated for administration to a subject suffering from and/or susceptible to FXS and/or other neurodevelopmental disorders. Such pharmaceutical compositions optionally comprise one or more additional therapeutically-active substances.

In accordance with some embodiments, the invention provides methods of treating FXS and/or other neurodevelopmental disorders comprising administering a pharmaceutical composition comprising at least one specific small molecule PAK inhibitor (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) to a subject in need thereof. In some embodiments, inventive small molecule PAK inhibitors are administered with other medications used to treat the symptoms of FXS. In some embodiments, the compositions are administered to humans.

The invention encompasses the preparation and/or use of pharmaceutical compositions comprising a specific small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) for treatment of FXS and/or other neurodevelopmental disorders as an active ingredient. In some embodiments, such a pharmaceutical composition consist of the small molecule PAK inhibitor alone, in a form suitable for administration to a subject, or the pharmaceutical composition comprises the small molecule PAK inhibitor and one or more pharmaceutically acceptable excipients, one or more additional ingredients, and/or a combination of these. In some embodiments, the small molecule PAK inhibitor is present in the pharmaceutical composition in the form of a physiologically acceptable ester and/or salt, such as in combination with a physiologically acceptable cation and/or anion.

Although the descriptions of pharmaceutical compositions provided herein are principally directed to pharmaceutical compositions which are suitable for administration to humans, such compositions are generally suitable for administration to animals of all sorts. Patients to which administration of the pharmaceutical compositions of the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Pharmaceutical compositions as described herein may be prepared by any method known or hereafter developed in the art of pharmaceutics. In general, such preparatory methods include the step of bringing the small molecule PAK inhibitor into association with one or more excipients and/or one or more other accessory ingredients, and then, if necessary and/or desirable, shaping and/or packaging the product into a desired single- or multi-dose unit.

In some embodiments, a pharmaceutical composition of the invention is prepared, packaged, and/or sold in bulk, as a single unit dose, and/or as a plurality of single unit doses. As used herein, a “unit dose” is discrete amount of the pharmaceutical composition comprising a predetermined amount of the small molecule PAK inhibitor. The amount of the small molecule PAK inhibitor is generally equal to the dosage of the small molecule PAK inhibitor which would be administered to a subject and/or a convenient fraction of such a dosage such as, for example, one-half or one-third of such a dosage.

The relative amounts of small molecule PAK inhibitor, pharmaceutically acceptable excipient(s), and/or any additional ingredients in a pharmaceutical composition of the invention will vary, depending upon the identity, size, and/or condition of the subject treated and further depending upon the route by which the composition is to be administered. By way of example, the composition comprises between 0.1% and 100% (w/w) small molecule PAK inhibitor.

In some embodiments, pharmaceutical formulations of the present invention additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients are optionally included in the inventive formulations. In some embodiments, excipients such as cocoa butter and suppository waxes, coloring agents, coating agents, sweetening, flavoring, and perfuming agents are present in the composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL), sucrose fatty acid esters, polyethylene glycol fatty acid esters (e.g. CREMOPHOR), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON, and EUXYL. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In some embodiments, blood brain barrier-permeable (BBB-permeable) nanoparticle formulations are useful, for example, when a therapeutic agent (e.g., a PAK inhibitor) is found to have low access to the central nervous system (CNS). Methods of producing BBB-permeable nanoparticle formulations include, but are not limited to, methods described in U.S. Pat. Nos. 6,117,454 and 7,025,991 (both of which are incorporated herein by reference).

Liquid dosage forms for oral and parenteral administration include, but are not limited to, pharmaceutically acceptable emulsions, microemulsions, solutions, suspensions, syrups and elixirs. In addition to the small molecule PAK inhibitor(s), liquid dosage forms, in some embodiments, comprise inert diluents such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof Besides inert diluents, oral compositions include adjuvants such as wetting agents, emulsifying and suspending agents, sweetening, flavoring, and perfuming agents. In certain embodiments for parenteral administration, a small molecule PAK inhibitor is mixed with solubilizing agents such as CREMOPHOR, alcohols, oils, modified oils, glycols, polysorbates, cyclodextrins, polymers, and combinations thereof.

In some embodiments, injectable preparations, for example, sterile injectable aqueous or oleaginous suspensions are formulated according to the known art using suitable dispersing or wetting agents and suspending agents. In some embodiments, a sterile injectable preparation is a sterile injectable solution, suspension or emulsion in a nontoxic parenterally acceptable diluent or solvent, for example, as a solution in 1,3-butanediol. Among the acceptable vehicles and solvents that are employed are water, Ringer's solution, U.S.P. and isotonic sodium chloride solution. In addition, sterile, fixed oils are conventionally employed as a solvent or suspending medium. For this purpose any bland fixed oil is employed including synthetic mono- or di-glycerides. In addition, fatty acids such as oleic acid are used in the preparation of injectables.

Injectable formulations are sterilized, for example, by filtration through a bacterial-retaining filter, or by incorporating sterilizing agents in the form of sterile solid compositions which is dissolved or dispersed in sterile water or other sterile injectable medium prior to use.

In order to prolong the effect of a drug, it is often desirable to slow the absorption of the drug from subcutaneous or intramuscular injection. In some embodiments, this is accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which are prepared, in some embodiments, by mixing the small molecule PAK inhibitors of this invention with suitable non-irritating excipients such as cocoa butter, polyethylene glycol, or a suppository wax which are solid at ambient temperature but liquid at body temperature and therefore melt in the rectum or vaginal cavity and release the small molecule PAK inhibitor.

Solid dosage forms for oral administration include capsules, tablets, pills, powders, and granules. In such solid dosage forms, the small molecule PAK inhibitor is mixed with at least one inert, pharmaceutically acceptable excipient such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and/or (i) lubricants such as talc, calcium stearate, magnesium stearate, solid polyethylene glycols, sodium lauryl sulfate, and mixtures thereof. In the case of capsules, tablets and pills, the dosage form, in some embodiments, comprises buffering agents.

In some embodiments, solid compositions of a similar type are employed as fillers in soft and hard-filled gelatin capsules using such excipients as lactose or milk sugar as well as high molecular weight polyethylene glycols and the like. In some embodiments, solid dosage forms of tablets, dragees, capsules, pills, and granules are prepared with coatings and shells such as enteric coatings. They optionally comprise opacifying agents and are of a composition that they release the small molecule PAK inhibitor(s) only, or preferentially, in a certain part of the intestinal tract, optionally, in a delayed manner. Examples of embedding compositions which are used, in some embodiments, include polymeric substances and waxes. In some embodiments, solid compositions of a similar type are employed as fillers in soft and hard-filled gelatin capsules using such excipients as lactose or milk sugar as well as high molecular weight polethylene glycols and the like.

In some embodiments, small molecule PAK inhibitors are in micro-encapsulated form with one or more excipients as noted above. In some embodiments, the solid dosage forms of tablets, dragees, capsules, pills, and granules are prepared with coatings and shells such as enteric coatings, release controlling coatings. In some embodiments, in such solid dosage forms the small molecule PAK inhibitor is admixed with at least one inert diluent such as sucrose, lactose or starch. In some embodiments, such dosage forms comprise, as is normal practice, additional substances other than inert diluents, e.g., tableting lubricants and other tableting aids such a magnesium stearate and microcrystalline cellulose. In some embodiments, capsules, tablets and pills comprise buffering agents. They optionally comprise opacifying agents and are of a composition that they release the small molecule PAK inhibitor(s) only, or preferentially, in a certain part of the intestinal tract, optionally, in a delayed manner. Examples of embedding compositions include polymeric substances and waxes.

In some embodiments, dosage forms for topical and/or transdermal administration of a composition of this invention include ointments, pastes, creams, lotions, gels, powders, solutions, sprays, inhalants and/or patches. Generally, a small molecule PAK inhibitor is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a small molecule PAK inhibitor to the body. Such dosage forms are prepared, for example, by dissolving and/or dispensing the small molecule PAK inhibitor in the proper medium. Alternatively or additionally, the rate is controlled by either providing a rate controlling membrane and/or by dispersing the small molecule PAK inhibitor in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. In some embodiments, intradermal compositions are administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, in some embodiments, conventional syringes are used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. In some embodiments, topically-administrable formulations comprise from about 1.0% to about 10% (w/w) small molecule PAK inhibitor, although the concentration of the small molecule PAK inhibitor may be as high as the solubility limit of the small molecule PAK inhibitor in the solvent. In some embodiments, formulations for topical administration comprise one or more of the excipients and/or small molecule PAK inhibitors described herein.

In some embodiments, a pharmaceutical composition of the invention is prepared, packaged, and/or sold in a formulation suitable for pulmonary administration via the buccal cavity. In some embodiments, such a formulation comprises dry particles which comprise the small molecule PAK inhibitor and which have a diameter in the range from about 0.5 μm to about 7 μm or from about 1 μm to about 6 μm. Such compositions are conveniently in the form of dry powders for administration using a device comprising a dry powder reservoir to which a stream of propellant may be directed to disperse the powder and/or using a self propelling solvent/powder dispensing container such as a device comprising the small molecule PAK inhibitor dissolved and/or suspended in a low-boiling propellant in a sealed container. Such powders comprise particles wherein at least 98% of the particles by weight have a diameter greater than 0.5 μm and at least 95% of the particles by number have a diameter less than 7 μm. Alternatively, at least 95% of the particles by weight have a diameter greater than 1 μm and at least 90% of the particles by number have a diameter less than 6 μm. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant constitutes 50% to 99.9% (w/w) of the composition, and the small molecule PAK inhibitor constitutes 0.1% to 20% (w/w) of the composition. In some embodiments, the propellant further comprises additional ingredients such as a liquid non-ionic and/or solid anionic surfactant and/or a solid diluent (which, in some embodiments, has a particle size of the same order as particles comprising the small molecule PAK inhibitor).

In some embodiments, pharmaceutical compositions of the invention formulated for pulmonary delivery provide the small molecule PAK inhibitor in the form of droplets of a solution and/or suspension. In some embodiments, such formulations are prepared, packaged, and/or sold as aqueous and/or dilute alcoholic solutions and/or suspensions, optionally sterile, comprising the small molecule PAK inhibitor, and are conveniently administered using any nebulization and/or atomization device. In some embodiments, such formulations comprise one or more additional ingredients including, but not limited to, a flavoring agent such as saccharin sodium, a volatile oil, a buffering agent, a surface active agent, and/or a preservative such as methylhydroxybenzoate. In some embodiments, droplets provided by this route of administration have an average diameter in the range from about 0.1 μm to about 200 μm.

Formulations described herein as being useful for pulmonary delivery are useful for intranasal delivery of a pharmaceutical composition of the invention. Another formulation suitable for intranasal administration is a coarse powder comprising the small molecule PAK inhibitor and having an average particle from about 0.2 μm to 500 μm. Such a formulation is administered in the manner in which snuff is taken, i.e. by rapid inhalation through the nasal passage from a container of the powder held close to the nares.

Formulations suitable for nasal administration, for example, comprise from about as little as 0.1% (w/w) and as much as 100% (w/w) of the small molecule PAK inhibitor, and comprise one or more of the excipients and/or additional ingredients described herein. In some embodiments, a pharmaceutical composition of the invention is prepared, packaged, and/or sold in a formulation suitable for buccal administration. Such formulations, for example, are in the form of tablets and/or lozenges made using conventional methods, and are, for example, 0.1% to 20% (w/w) small molecule PAK inhibitor, the balance comprising an orally dissolvable and/or degradable composition and, optionally, one or more of the excipients and/or additional ingredients described herein. Alternately, formulations suitable for buccal administration comprise a powder and/or an aerosolized and/or atomized solution and/or suspension comprising the small molecule PAK inhibitor. In some embodiments, such powdered, aerosolized, and/or aerosolized formulations, when dispersed, have an average particle and/or droplet size in the range from about 0.1 μm to about 200 μm, and comprise one or more of the excipients and/or additional ingredients described herein.

In some embodiments, a pharmaceutical composition of the invention is prepared, packaged, and/or sold in a formulation suitable for ophthalmic administration. Such formulations, for example, are in the form of eye drops including, for example, a 0.1%/1.0% (w/w) solution and/or suspension of the small molecule PAK inhibitor in an aqueous or oily liquid excipient. In some embodiments, such drops comprise buffering agents, salts, and/or one or more other of the excipients and/or additional ingredients described herein. Other opthalmically-administrable formulations which are useful include those which comprise the small molecule PAK inhibitor in microcrystalline form and/or in a liposomal preparation. Ear drops and/or eye drops are contemplated as being within the scope of this invention.

General considerations in the formulation and/or manufacture of pharmaceutical agents are found, for example, in Remington: The Science and Practice of Pharmacy 21^{st} ed., Lippincott Williams & Wilkins, 2005 (incorporated herein by reference).

### Administration

In accordance with the present invention, specific small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) are useful in the treatment of FXS and/or other neurodevelopmental disorders. Thus, in one aspect, pharmaceutical compositions containing one or more specific small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) are administered to one or more individuals suffering from, susceptible to, and/or exhibiting symptoms of FXS. The present invention therefore encompasses methods of inhibiting PAK activity, as well as methods of treating FXS and/or other neurodevelopmental disorders in subjects.

In some embodiments, a therapeutically effective amount of a pharmaceutical composition comprising a specific small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) is delivered to a subject and/or organism prior to, simultaneously with, and/or after diagnosis with FXS and/or other neurodevelopmental disorder. In some embodiments, a therapeutic amount of a pharmaceutical composition is delivered to a patient and/or organism prior to, simultaneously with, and/or after onset of symptoms of FXS and/or other neurodevelopmental disorder. In some embodiments, the amount of pharmaceutical composition is sufficient to treat, alleviate, ameliorate, relieve, delay onset of, inhibit progression of, reduce severity of, and/or reduce incidence of one or more symptoms or features of FXS and/or other neurodevelopmental disorder.

In some embodiments, pharmaceutical compositions, according to the method of the present invention, are administered using any amount and any route of administration effective for treatment. The exact amount required will vary from subject to subject, depending on the species, age, and general condition of the subject, the severity of the infection, the particular composition, its mode of administration, its mode of activity, and the like. Compositions of the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disorder being treated and the severity of the disorder; the activity of the specific small molecule PAK inhibitor employed; the specific composition employed; the age, body weight, general health, sex and diet of the subject; the time of administration, route of administration, and rate of excretion of the specific small molecule PAK inhibitor employed; the duration of the treatment; drugs used in combination or coincidental with the specific small molecule PAK inhibitor employed; and like factors.

In some embodiments, pharmaceutical compositions of the present invention are administered by any route. In some embodiments, pharmaceutical compositions of the present invention are administered by a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, and/or drops), transdermal, mucosal, nasal, buccal, enteral, sublingual; by intratracheal instillation, bronchial instillation, and/or inhalation; and/or as an oral spray, nasal spray, and/or aerosol. In some embodiments, inventive compositions are administered parenterally. In some embodiments, inventive compositions are administered intravenously. In some embodiments, inventive compositions are administered orally.

In general the most appropriate route of administration will depend upon a variety of factors including the nature of the composition (e.g., its stability in the environment of the gastrointestinal tract), the condition of the subject (e.g., whether the subject is able to tolerate oral administration), etc.

In certain embodiments, a composition is administered in amounts ranging from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. In some embodiments, a composition is administered in amounts ranging from about 0.01 mg to about 1 mg per kilogram of body weight. In some embodiments, the desired dosage is delivered three times a day, two times a day, once a day, every other day, every third day, every week, every two weeks, every three weeks, and/or every four weeks. In certain embodiments, the desired dosage is delivered using multiple administrations (e.g., two, three, four, five, six, seven, eight, nine, ten, eleven, twelve, thirteen, fourteen, or more administrations).

It will be appreciated that small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof) and pharmaceutical compositions are employed, in some embodiments, in combination therapies in accordance with the present invention. In some embodiments, the present invention encompasses “therapeutic cocktails” comprising specific small molecule PAK inhibitors (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof). The particular combination of therapies (therapeutics or procedures) to employ in a combination regimen will take into account compatibility of the desired therapeutics and/or procedures and the desired therapeutic effect to be achieved. In some embodiments, the therapies employed achieve a desired effect for the same purpose. For example, a small molecule PAK inhibitor is administered with another agent that is used to treat symptoms of FXS and/or other neurodevelopmental disorder. In some embodiments, the therapies employed achieve different effects (e.g., control of any adverse side effects).

In some embodiments, pharmaceutical compositions of the present invention are administered either alone or in combination with one or more other therapeutic agents. By “in combination with,” it is not intended to imply that the agents must be administered at the same time and/or formulated for delivery together, although these methods of delivery are within the scope of the invention. In some embodiments, compositions are administered concurrently with, prior to, or subsequent to, one or more other desired therapeutics or medical procedures. In general, each agent will be administered at a dose and/or on a time schedule determined for that agent. Additionally, the invention encompasses the delivery of the inventive pharmaceutical compositions in combination with agents that improve their bioavailability, reduce and/or modify their metabolism, inhibit their excretion, and/or modify their distribution within the body.

The particular combination of therapies (therapeutics and/or procedures) to employ in a combination regimen will take into account compatibility of the desired therapeutics and/or procedures and/or the desired therapeutic effect to be achieved. In some embodiments, therapies employed achieve a desired effect for the same disorder (for example, an inventive agent may be administered concurrently with another therapeutic agent used to treat the same disorder), and/or they achieve different effects (e.g., control of any adverse side effects). In some embodiments, compositions of the invention are administered with a second therapeutic agent that is approved by the U.S. Food and Drug Administration.

The therapeutically active agents utilized in combination are either administered together in a single composition or administered separately in different compositions.

In general, it is expected that agents utilized in combination with be utilized at levels that do not exceed the levels at which they are utilized individually. In some embodiments, the levels utilized in combination will be lower than those utilized individually.

In some embodiments, the pharmaceutical compositions of the present invention are administered alone and/or in combination with other agents that are used to treat the symptoms of FXS. In some embodiments, such agents treat seizures and/or mood instability, including but not limited to as Carbamazepine (TEGRETOL®), Valproic Acid, Divalproex (DEPAKOTE®), Lithium Carbonate, Gabapentin (NEURONTIN®), Lamotrigine (LAMICTAL®), Topiramate (TOPAMAX®), Tiagabine (GABITRIL®), Vigabatrin (SABRIL®), Phenobarbital, Primidone (MYSOLINE®), and/or Phenytoin (DILANTIN®).

In some embodiments, such agents are central nervous system stimulants, including but not limited to Methylphenidate (RITALIN®), Dextroamphetamine (DEXEDRINE®; ADDERALL®), Ditropan (CONCERTA®), L-acetylcarnitine, Venlafaxine (EFFEXOR®), Nefazodone (SERZONE®), Amantadine (SYMMETREL®), Buproprion (WELLBUTRIN®), Desipramine, Imipramine, and/or Buspirone (BUSPAR®).

In some embodiments, such agents are antihypertensive drugs, including but not limited to Clonidine (CATAPRES®) and/or Guanfacine (TENEX®).

In certain embodiments, such agents include folic acid.

In some embodiments, such agents are selected serotonin reuptake inhibitors, including but not limited to Fluoxetine (PROZAC®), Sertraline (ZOLOFT®), and/or Citalopram (CELEXA®).

In some embodiments, such agents are antipsychotics, including but not limited to Risperidone (RISPERIDAL®), Olanzepine (ZYPREXA®), and/or Quetiapine (SEROQUEL®).

In certain embodiments, such agents are used to treat sleep disturbances, including but not limited to Desyrel (TRAZODONE®) and/or Melatonin.

In some embodiments, therapeutically active agents utilized in combination are administered together in a single composition or administered separately in different compositions.

In some embodiments, small molecule PAK inhibitors are administered in combination with one or more other PAK inhibitors in accordance with the present invention.

One of ordinary skill in the art will understand that the examples presented above are not meant to be limiting. The principles presented in the examples above can be generally applied to any combination therapies for treatment of FXS and/or other neurodevelopmental disorders.

### Kits

The invention provides a variety of kits comprising one or more small molecule PAK inhibitors. For example, the invention provides kits comprising at least one small molecule PAK inhibitor and instructions for use. In some embodiments, a kit comprises multiple different small molecule PAK inhibitors. In some embodiments, a kit comprises any of a number of additional components or reagents in any combination. All of the various combinations are not set forth explicitly but each combination is included in the scope of the invention.

According to certain embodiments of the invention, a kit includes, for example, (i) a small molecule PAK inhibitor (e.g. BMS-387032; SNS-032; CHI4-258; TKI-258; EKB-569; JNJ-7706621; PKC-412; staurosporine; SU-14813; sunitinib; VX-680; MK-0457; combinations thereof; and/or derivatives analogs or thereof); (ii) instructions for administering the small molecule PAK inhibitor to a subject suffering from, susceptible to, and/or exhibiting symptoms of FXS and/or other neurodevelopmental disorder.

Kits typically include instructions for use of inventive small molecule PAK inhibitors. Instructions, for example, comprise protocols and/or describe conditions for production of small molecule PAK inhibitors, administration of small molecule PAK inhibitors to a subject in need thereof, etc. Kits generally include one or more vessels or containers so that some or all of the individual components and reagents may be separately housed. In some embodiments, kits also include a means for enclosing individual containers in relatively close confinement for commercial sale, e.g., a plastic box, in which instructions, packaging materials such as styrofoam, etc., are enclosed. In some embodiments, an identifier, e.g., a bar code, radio frequency identification (ID) tag, etc., is present in or on the kit or in or one or more of the vessels or containers included in the kit. In some embodiments, an identifier is used, e.g., to uniquely identify the kit for purposes of quality control, inventory control, tracking, movement between workstations, etc.

### Exemplification

The representative Examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following Examples contain important additional information, exemplification and guidance that can be adapted to the practice of this invention in its various embodiments and the equivalents thereof. It will be appreciated, however, that these examples do not limit the invention. Variations of the invention, now known and/or further developed, are considered to fall within the scope of the present invention as described herein and as hereinafter claimed.

Previous studies have consistently associated mental retardation with abnormalities in the number and size of synapses. In FXS patients and in FMR1 KO mice, cortical neurons display more postsynaptic dendritic spines and a higher proportion of longer and thinner spines compared to normal individuals. The FMR1 KO mice exhibit impaired cortical LTP compared to wild-type mice. Strikingly, the abnormalities in cortical synaptic morphology and plasticity of FMR1 KO mice are opposite to those we observed in transgenic (dnPAK TG) mice in which activity of p21-activated kinase (PAK) is inhibited by its dominant negative form (dnPAK), specifically in the postnatal forebrain. In the dnPAK TG mice, we found that cortical neurons display fewer dendritic spines and a lower proportion of longer and thinner spines compared to wild type mice (Hayashi et al., 2004, Neuron, 42:773; incorporated herein by reference). These transgenic mice exhibit enhanced cortical LTP, in contrast to the impaired cortical LTP in FMR1 KO mice. One hypothesis that may explain these results is that signaling pathways mediated by PAK and FMR1 may antagonize each other to regulate synaptic morphology and function, and this hypothesis was tested in the following examples.

### EXAMPLE 1

### PAK Inhibition Rescues Spine Morphological Abnormalities in FMR1 KO Mice

### Materials and Methods

Golgi Analysis

Following the Golgi-Cox technique (Ramon-Moliner, 1970, Contemporary Research Methods in Neuroanatomy, Springer, Berlin, Heidelberg, New York), 120 μm thick serial sections were obtained from brains of two-month-old male littermates. Slides containing these sections were coded before quantitative analysis, and the code was broken only after the analysis was completed. Layer II/III pyramidal neurons in the temporal cortex were visualized under Olympus upright BX61 with motorized XY stage using Neurolucida/stereology software (Microbrightfield). On each primary apical dendritic branch, ten consecutive 10 μm-long dendritic segments were analyzed to quantify spine density. To ensure sampling consistency among Golgi analysis and electrophysiology experiments, analyses in the temporal cortex were all carried out in slices or sections corresponding to FIG. 62-67 of the mouse brain atlas (Franklin et al., The mouse brain in stereotaxic coordinates, Academic, San Diego, Calif., 1997).

Electron Microscopy

Two-month-old male littermates were anaesthetized and perfused according to standard procedures. Blocks of temporal cortex were then embedded, from which 1 μm thick sections were cut and stained with 1% toluidine blue to guide the further trimming to isolate layer II/III of temporal cortex. 90 nm ultrathin sections were then cut and stained with uracyl acetate and lead citrate. Randomly selected neuropil areas were photographed at a 10,000× magnification with a JEOL 1200EX electron microscope. Image negatives were scanned at 1200 dpi and analyzed by OpenLab Program (Improvision). Excitatory synapses bearing spines were defined by the presence of a clear post-synaptic density (PSD) facing at least three presynaptic vesicles. Micrographs covering 500 μm²-1000 μm² neuropil regions from each mouse were analyzed and used for quantitation. PSD length and percentage of perforated synapses were quantified from the same population of synapses. The measurements were all performed by an experimenter blind to the genotype.

### Results

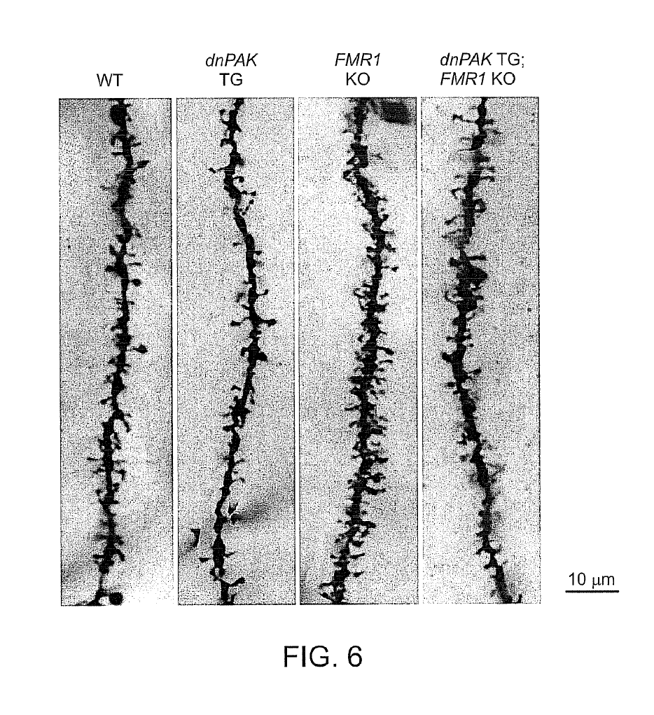

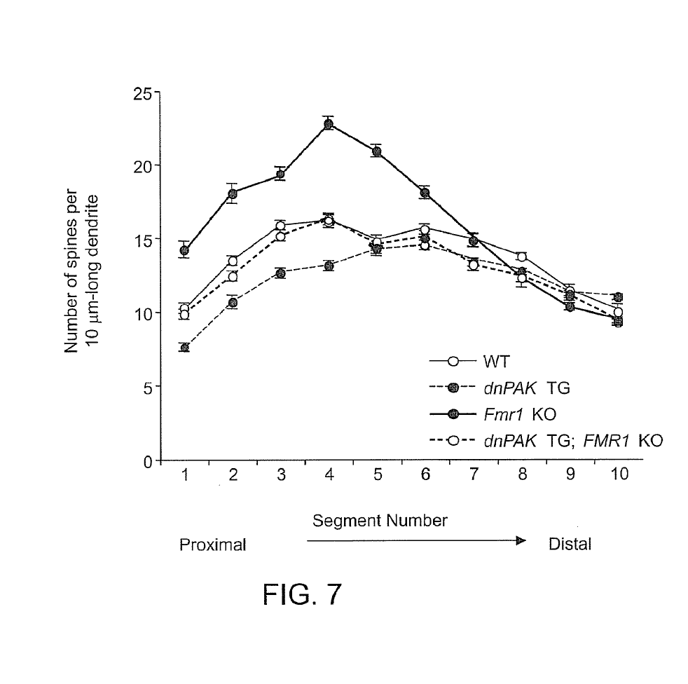

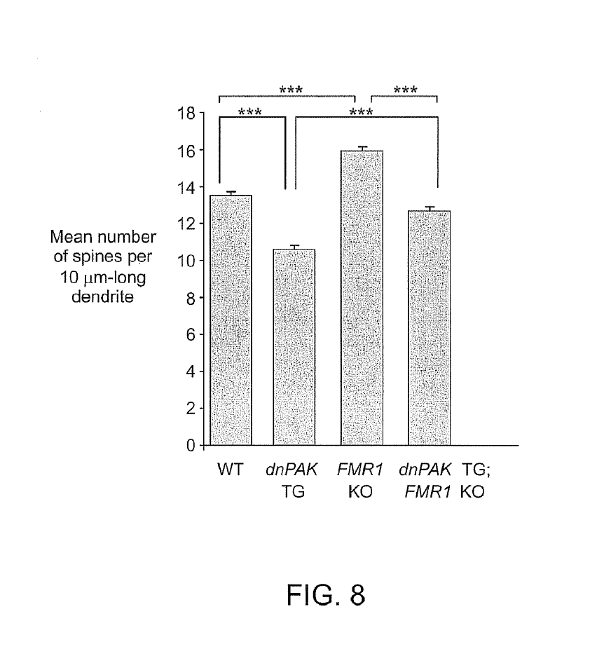

Since spine abnormality is a pathological hallmark in FXS patients and FMR1 KO mice at the cellular level, dendritic spine morphology was examined by measuring spine density in apical dendrites of Golgi-stained layer II/III pyramidal neurons of the temporal cortex of dMT mice as well as their littermates, dnPAK TG, FMR1 KO, and wild-type mice. The number of spines per 10 μm of dendritic segments that run proximal to distal to the neuronal soma was quantified. In proximal dendritic segments, spine density was lower in dnPAK TG mice compared to wild-type mice while it was higher in FMR1 KO mice compared to wild-type mice (FIGS. 6 and 7). In contrast, spine density in dMT mice was comparable to that in wild-type controls in all dendritic segments except segments 7 and 8 (FIG. 7). When averaged over all segments, mean spine density in dMT mice was significantly lower than that in FMR1 KO mice and significantly higher than that in dnPAK TG mice (FIG. 8). These results indicate that PAK inhibition partially restores the abnormality of spine density in FMR1 KO mice.

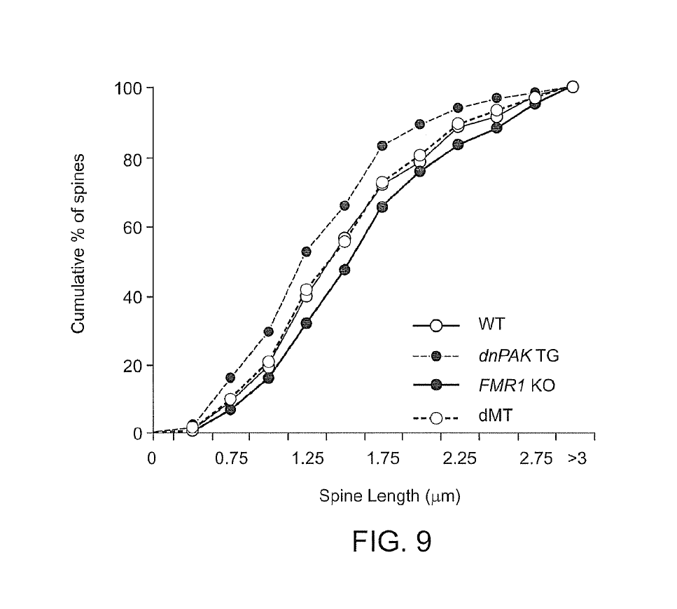

In addition to in increased spine density, cortical neurons from FXS patients and FMR1 KO mice exhibit increased spine length (Hinton et al., 1991, Am. J. Med. Genet., 41:289; Comery et al., 1997, Proc. Natl. Acad. Sci., USA, 94:5401; Irwin et al., 2001, Am. J. Med. Genet., 98:161; and McKinney et al., 2005, Am. J. Med. Genet. B. Neuropsychiatr. Genet., 136:98; all of which are incorporated herein by reference). To investigate whether dnPAK can also restore this abnormality, spine length (the radial distance from tip of spine head to dendritic shaft) of Golgi-stained pyramidal neurons in the four genotypes was measured. In cumulative frequency plots, FMR1 KO neurons exhibited a significant shift in the overall spine distribution towards spines of longer length compared to wild-type neurons, while dnPAK TG neurons exhibited the opposite shift to shorter spines (FIG. 9). In contrast, spine length distribution of dMT neurons overlapped well with that of wild-type neurons (FIG. 9), indicating that PAK inhibition is sufficient to restore the cortical spine length abnormality in FMR1 KO mice.

### EXAMPLE 2

### PAK Inhibition Rescues Reduced Cortical LTP in FMR1 KO Mice

### Materials and Methods

Electrophysiology

From three-month-old male littermates, coronal brain slices containing temporal cortex were prepared and left to recover for at least 1 hour before recording in oxygenated (95% O₂ and 5% CO₂) warm (30° C.) artificial cerebrospinal fluid containing 124 mM NaCl, 5 mM KCl, 1.25 mM NaH₂PO₄, 1 mM MgCl₂, 2 mM CaCl₂, 26 mM NaHCO₃, and 10 mM dextrose. Field potentials (FPs) in layer II/III evoked by layer IV stimulation were measured as previously described (Hayashi et al., 2004, Neuron 42:773) and responses were quantified as the amplitude of FP in cortex. LTP was induced by TBS, which consisted of eight brief bursts (each with 4 pulses at 100 Hz) of stimuli delivered every 200 msec.\

### Results

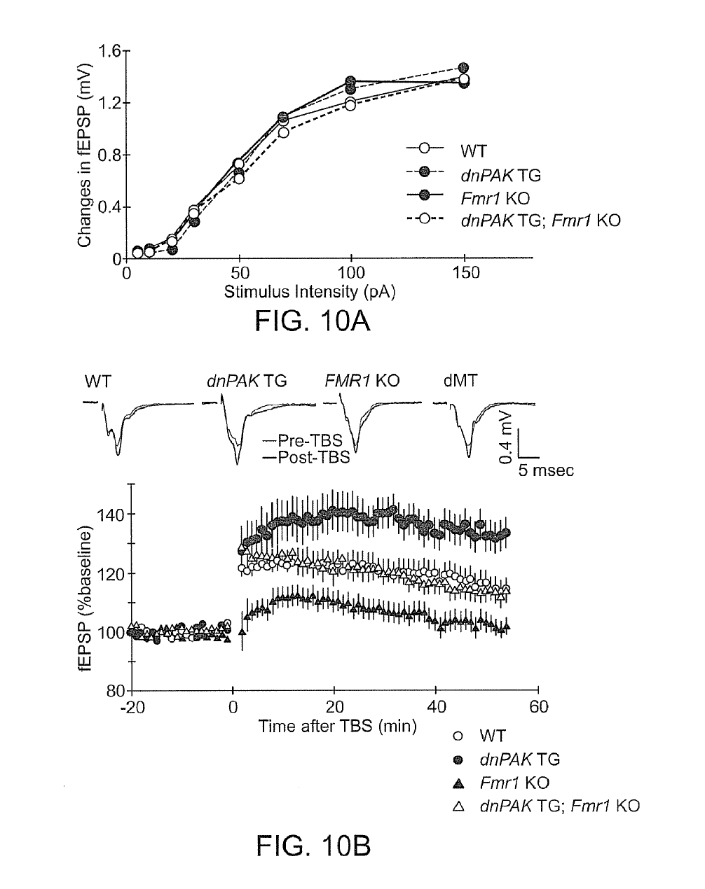

Cortical long-term potentiation (LTP) has been shown to be reduced in FMR1 KO mice while it is enhanced in dnPAK TG mice (Li et al., 2002, Mol. Cell. Neurosci., 19:138; Zhao et al., 2005, J. Neurosci., 25:7385; and Hayashi et al., 2004, Neuron, 42:773; all of which are incorporated herein by reference). To assess the effect of PAK inhibition on the cortical synaptic transmission and plasticity in FMR1 KO mice, extracellular field recordings in temporal cortex layer II/III synapses were carried out while stimulating layer IV. Basal synaptic transmission, as measured by field potential responses to a range of stimulus intensities, did not differ between the four genotypes (FIG. 10A). However, as expected, administration of theta-burst stimulation (TBS) at 100 Hz produced LTP of a lower magnitude in FMR1 KO mice than in wild-type mice and LTP of a higher magnitude in dnPAK TG than in wild-type mice (FIG. 10B). In contrast, the magnitude of LTP was indistinguishable between dMT mice and wild-type controls at various times following the application of the stimulus (FIG. 10B). This demonstrates that PAK inhibition rescues LTP defects in FMR1 KO mice.

### EXAMPLE 3

### PAK Inhibition Rescues Multiple Behavioral Defects in FMR1 KO Mice

### Materials and Methods

Open Field Test

Two-month-old male littermates were subjected to the open field test according to standard procedures. Each mouse ran for 10 minutes in a VersaMax activity monitor chamber (Accuscan Instruments). Open field activity was detected by photobeam breaks and analyzed by the VersaMax software. Stereotypy is recorded when the mouse breaks the same beam (or set of beams) repeatedly. Stereotypy count is the number of beam breaks that occur during this period of sterotypic activity.

Trace Fear Conditioning Task

Three-month-old male littermates were subjected to trace fear conditioning as previously described (Zhao et al., 2005, J. Neurosci., 25:7385). On day 1, mice were placed in the training chamber (Chamber A, Coulbourn Instruments) for 60 seconds before the onset of a 15-second white noise tone (conditioned stimulus or CS). 30 seconds later, mice received a 1-second shock (0.7 mA intensity; unconditioned stimulus or US). Thus, one trial is composed of tone (CS), 30 seconds blank time (also called “trace”), and then shock (US). Seven trials with an intertrial interval (ITI) of 210 seconds were performed to let the mice learn the association between tone and shock across a time gap. To examine whether mice remember this association, on day 2, mice were placed into a new chamber (Chamber B) with a different shape and smell from those in Chamber A. After 60 seconds, a 15-second tone was repeated seven times with an ITI of 210 seconds. Video images were digitized and the percentage of freezing time during each ITI was analyzed by Image FZ program (O'Hara & Co). Freezing was defined as the absence of all but respiratory movement for a 1-second period.

### Results

PAK Inhibition Rescues Multiple Behavioral Defects in FMR1 KO Mice

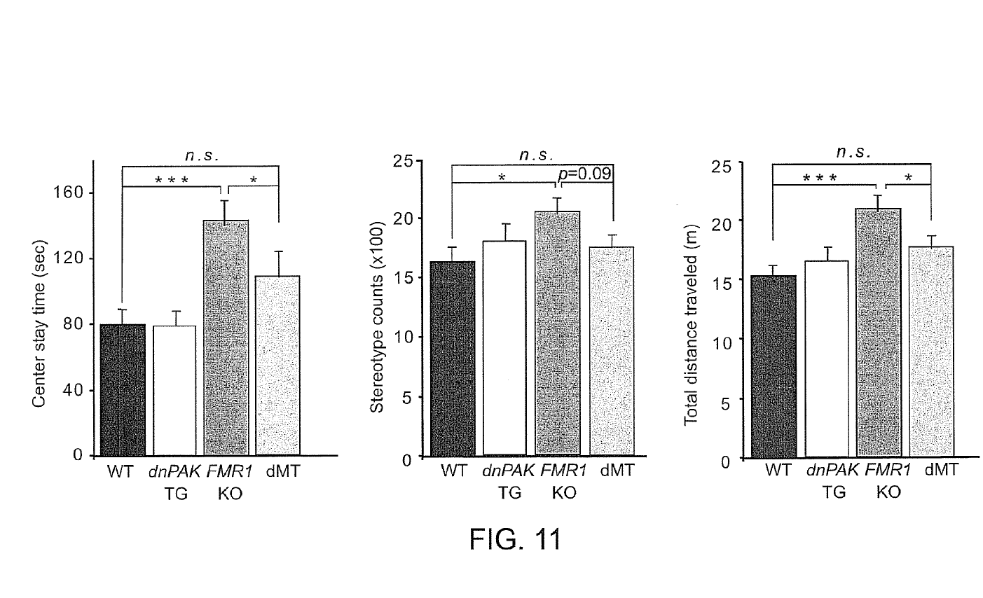

To test if the partial rescue of spine morphology and the complete rescue of cortical LTP by PAK inhibition could ameliorate behavioral deficits present in FMR1 KO mice, the mice of various genotypes were subjected to a series of behavioral tasks. In an open field test where mice are placed in a box and allowed to run freely for ten minutes, FMR1 KO mice exhibited three abnormal behaviors compared to wild-type mice (Peier et al., 2000, Hum. Mol. Genet., 9:1145). (1) Hyperactivity: they traveled a longer distance and moved for a longer period of time (FIG. 11); (2) Stereotypy: they exhibited a higher number of repetitive behaviors (FIGS. 11); and (3) Hypo-anxiety: they stayed in the center field for a longer period of time and in the corners of the field for a shorter period of time (FIG. 11). In all three behaviors, dMT mice exhibited performance comparable to wild-type controls (FIG. 11). This indicated that PAK inhibition in FMR1 KO mice restores locomotion, repetitive behavior, and anxiety to wild-type levels.

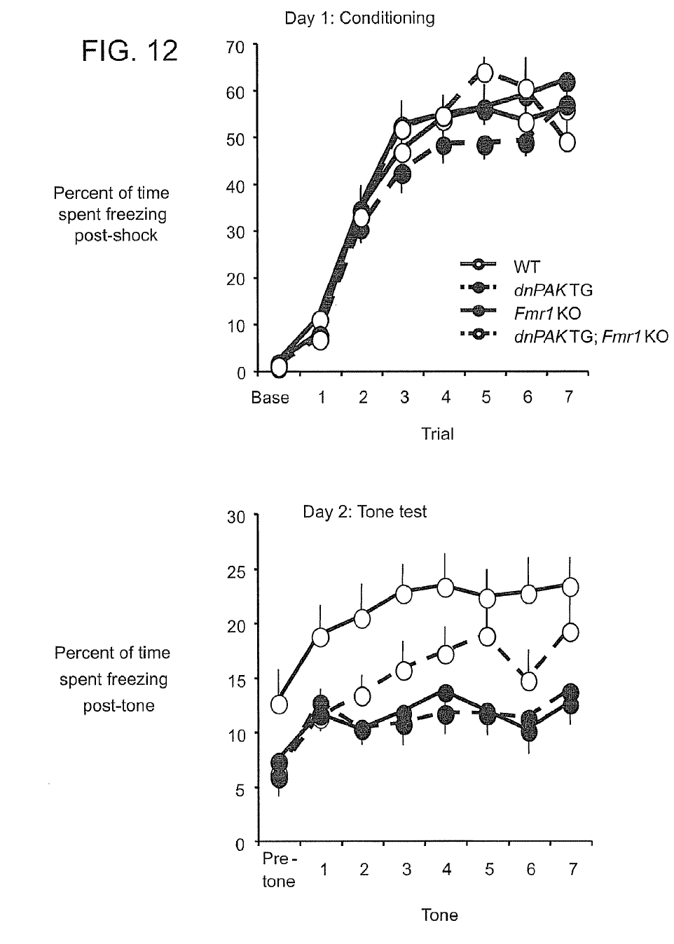

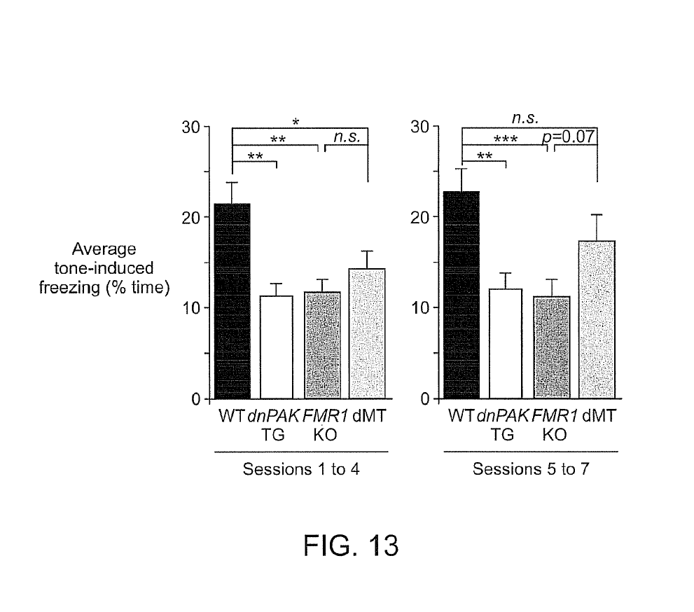

To further examine whether PAK inhibition can rescue abnormal cortex-dependent behaviors, trace fear conditioning was performed, which is a test that depends on the integrity of the prefrontal cortex and is sensitive to attention-distracting stimuli (McEchron et al., 1998, Hippocampus, 8:638; and Han et al., 2003, Proc. Natl. Acad. Sci., USA, 100:13087; both of which are incorporated herein by reference). It was previously shown that FMR1 KO mice are impaired in this form of conditioning, which may relate to the attention deficits in FXS patients (Zhao et al., 2005, J. Neurosci., 25:7385; incorporated herein by reference). In this task, a conditioning trial was composed of a tone (as the conditioned stimulus or CS), then a 30-second time gap (also called “trace”) and finally an electric shock (as the unconditioned stimulus or US). Seven trials were given to allow the mice to learn the association between the tone and the shock across the 30-second time gap. Mice that learn and remember this association will become immobile (or “freeze”) in response to the tone, even when they are placed into a new chamber with a different shape and smell compared to the training chamber. During training, the four genotypes exhibited comparable amounts of freezing in all conditioning trials (FIG. 12), suggesting normal memory acquisition. However, when placed in a new chamber 24 hours after training, both FMR1 KO mice and dnPAK TG mice exhibited a significant reduction in tone-induced freezing compared to wild-type controls (FIG. 12), indicating an impaired trace fear memory in these two genotypes. dMT mice also showed freezing deficits during the first several tone sessions (sessions 1 to 4) compared to wild-type controls (FIG. 12), although the deficits during these sessions were, on average, less pronounced compared to dnPAK TG or FMR1 KO mice (FIG. 13). However, with additional tone sessions (sessions 5 to 7), freezing by dMT caught up to that of wild-type while its difference from FMR1 KO mice almost reached statistical significance (p=0.07, FIG. 13). Thus, dMT mice are slow in expressing the memory and/or require a repetition of the recall cue (tone), but they can eventually (after 5 tone sessions) recall the memory at the level that is not significantly different from the wild-type level.

### EXAMPLE 4

### PAK1 and FMRP Physically Interact

### Materials and Methods

Animal Handling, Experimental Design, and Data Analysis

All strains of mice are of the C57B6 background. FMR1 KO mice were obtained from Dr. Steven Warren. dnPAK TG mice were generated previously (Hayashi et al., 2004, Neuron, 42:773). Mouse maintenance and all experimental procedures were performed in compliance with National Institute of Health guidelines. All experiments were conducted in a blind fashion. Unless specified otherwise, data were analyzed with Statview software (SAS) using one-way ANOVA test followed by Fisher's protected least significance difference (PLSD) post hoc test. Values are presented as mean±SEM.

Immunoprecipitation and Western Blotting

Mouse brains were homogenized in ice-cold homogenization buffer (0.32 M sucrose; 10 mM Tris-HCl, pH 7.4; 5 mM EDTA; Complete Protease Inhibitor Cocktail Tablets (Roche)) and centrifuged at 1,000×g for 10 minutes at 4° C. The supernatant was collected and centrifuged at 21,000×g for 15 minutes at 4° C. The pellet was resuspended in TE buffer (10 mM Tris-HCl pH 7.4; 5 mM EDTA) and one-ninth volume of cold DOC buffer (500 mM Tris-HCl, pH 9.0; 10% sodium deoxycholate) was added. The mixture was incubated in a 37° C. water bath for 30 minutes while shaking and mixed with one-ninth volume of Buffer T (1% Triton X-100; 1% sodium deoxycholate; 500 mM Tris-HCl, pH 9.0). The membrane extract was dialyzed against binding/dialysis buffer (50 mM Tris-HCl, pH 7.4; 0.1% Triton X-100) at 4° C. overnight.

For immunoprecipitation, the dialyzed membrane extract was pre-cleared with protein A-sepharose beads, then incubated with α-PAK1 (N-20 from Santa Cruz Biotech) or control rabbit serum (Sigma) in binding/dialysis buffer for 3 hours, and then incubated with protein A-sepharose beads overnight at 4° C. To test binding specificity, α-PAK1 was also incubated with its corresponding blocking peptide (Santa Cruz Biotech) prior to incubation with the membrane extract. The beads were washed three times with binding/dialysis buffer.

Proteins that bound to the beads were separated by SDS-PAGE, transferred to a nitrocellulose membrane, and subjected to western blot analysis. For PAK1 western blots, the membrane was blocked in 10% milk, then incubated with α-PAK1 antibody diluted at 1:1000, then incubated with a-rabbit horse radish peroxidase (HRP, Sigma) diluted at 1:1000, and then developed with enhanced chemiluminescence (ECL) Renaissance kit (New England Nuclear). For FMRP western blots, the membrane was processed with the Blast blotting amplification system (Perkin Elmer) with α-FMRP antibody (Chemicon) diluted at 1:1000, biotinylated α-mouse diluted at 1:1000, and streptavidin-HRP diluted at 1:1000.

GST Pull-Downs

GEX6p-1 plasmid encoding GST was purchased from Pharmacia. GST-PAK1 plasmid was obtained from Dr. Joe Kissil (Kissil et al., 2003, Mol. Cell, 12:841; incorporated herein by reference). Plasmids encoding FMRP and its mutants were obtained from Dr. Edouard Khandjian (Mazroui et al., 2003, Hum. Mol. Genet., 12:3087; incorporated herein by reference). GST and GST-PAK1 proteins were expressed in BL21 E. coli, purified on glutathione sepharose 4B (GS4B) beads (Pharmacia), and dialyzed with PBS overnight. FMRP and its mutants were in vitro-translated with the TNT coupled reticulocyte lysate systems kit (Promega) and labeled with Transcend tRNA (Promega). GST or GST-PAK1 was incubated with FMRP or its mutants in binding buffer (50 mM Tris-HCl, pH 7.5; 120 mM NaCl; 10 mM MgCl₂; 5% glycerol; 1% Triton X-100) for 3 hours. GS4B beads were added and incubated for 1 hour. The beads were washed three times with the binding buffer. Proteins that bound to the beads were separated by SDS-PAGE, transferred to a nitrocellulose membrane, and subjected to western blot analysis. To detect in vitro-translated FMRP or its mutants, the membrane was blocked, incubated with streptavidin-HRP, washed, and developed with ECL Renaissance kit.

### Results

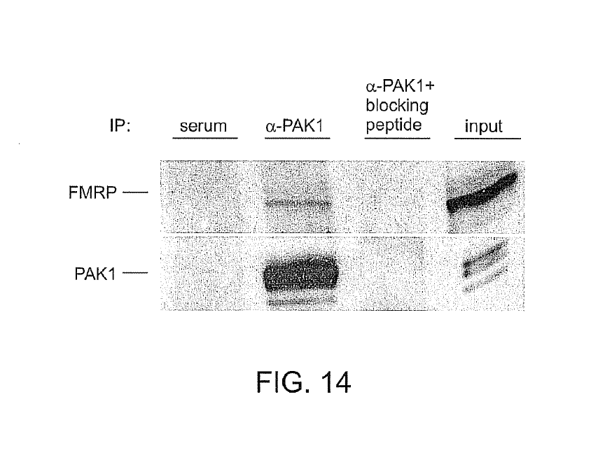

The morphological, electrophysiological and behavioral data presented in Examples 1-3 demonstrate that PAK inhibition rescues (at least partially) multiple abnormalities in FMR1 KO mice. To begin to understand the underlying mechanism, it was determined whether PAK1 and FMRP physically interact via immunoprecipitation followed by Western blot analysis. Since PAK1 and FMRP are both localized in synapses (Weiler et al., 1997, Proc. Natl. Acad. Sci., USA, 94:5395; and Hayashi et al., 2004, Neuron, 42:773; both of which are incorporated herein by reference), synapse-enriched membrane extract was prepared from mouse brain and subjected the extract to immunoprecipitation with a PAK1 antibody (α-PAK1). Proteins that may co-precipitate through their direct or indirect interaction with PAK1 were separated by SDS-PAGE and subjected to Western blot analysis with an FMRP antibody. FMRP immuno-reactivity was observed in PAK1 immunoprecipitates but not in control serum immunoprecipitates (FIG. 14). This interaction is specific because it did not occur when PAK1 antibody was pre-incubated with a blocking peptide, which competes with PAK1 for binding to the PAK1 antibody, prior to immunoprecipitation (FIG. 14). This result shows that the endogenous PAK1 and FMRP interact, directly or indirectly, in the brain.

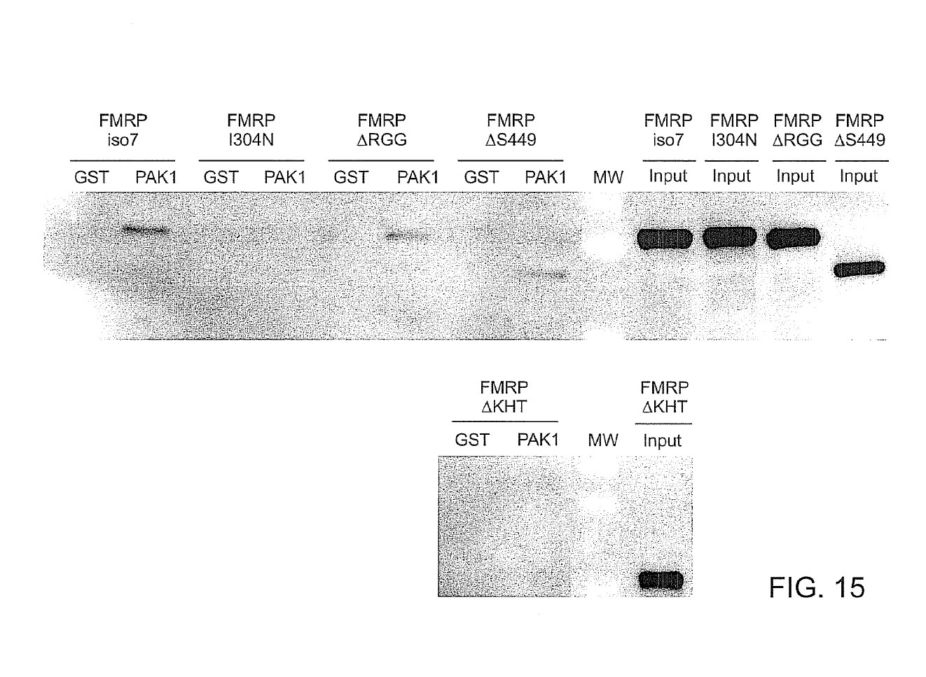

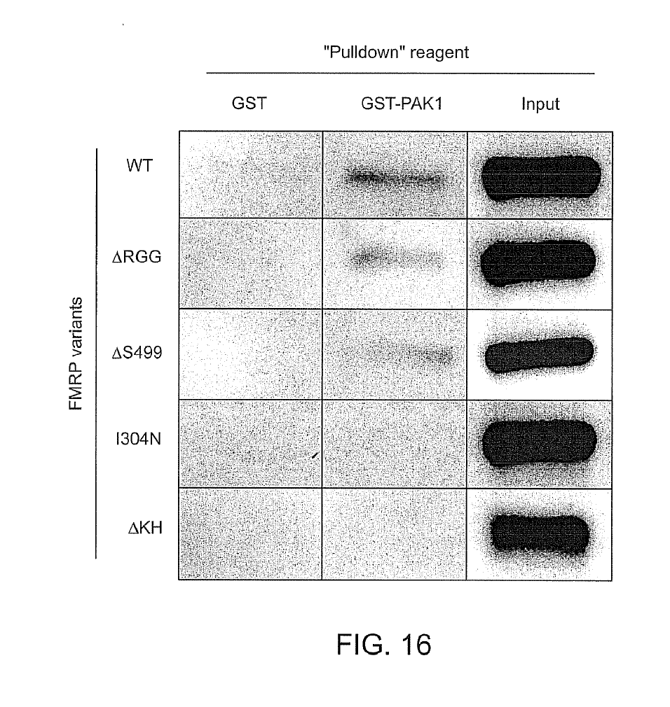

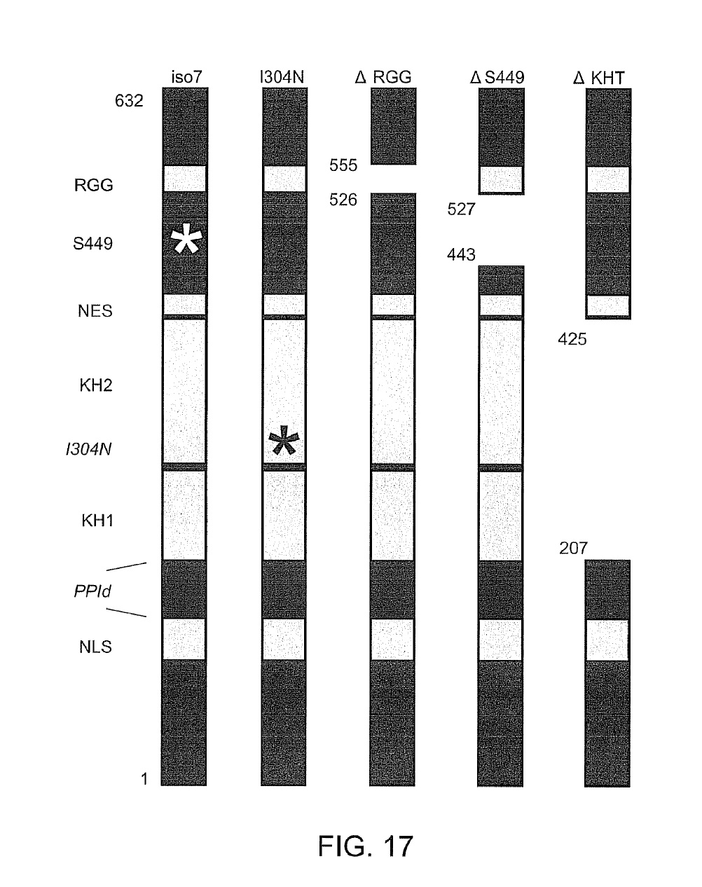

To examine whether PAK1 directly interacts with FMRP, a glutathione S-transferase (GST)-pull down assay was performed in which in vitro-translated FMRP was incubated with either GST or GST-tagged PAK1 (GST-PAK1). GST-PAK1, but not GST alone, bound to FMRP (FIGS. 15 and 16), suggesting a direct interaction between PAK1 and FMRP. FMRP contains a primary phosphorylation site at Ser 499 and three RNA-binding domains (KH1, KH2 and RGG) that are conserved among species (FIG. 17; O'Donnell and Warren, 2002, Annu. Rev. Neurosci., 25:315; and Ceman et al., 2003, Hum. Mol. Genet., 12:3295; both of which are incorporated herein by reference). To map the PAK1-binding region on FMRP, a series of deletion or point mutants of FMRP were used in the GST-pull down assay. An FMRP mutant without the RGG box (ΔRGG) or phosphorylation domain containing Ser 499 (ΔS499) was still able to bind to PAK1, whereas an FMRP mutant without KH domains (ΔKH) or with a point mutation in the KH2 domain previously found in a human with severe FXS (I304N; Feng et al., 1997, Mol. Cell, 1:109) was unable to bind to PAK1 (FIGS. 16 and 17). These results show that PAK1 directly binds to FMRP and this interaction requires the integrity of the KH domains of FMRP.

### EXAMPLE 5

### Identification of Compounds Having High Affinity for PAK Active Sites

The present example describes the identification of small molecule compounds that have high affinity for the active site of one or more PAK kinases. A competitive binding assay was utilized, which was developed by Ambit, Inc. (San Diego, Calif.), comprising three components: (1) an immobilized kinase “bait” probe (e.g., staurosporine) having high affinity for the catalytic site of multiple kinases; (2) full length PAK or a PAK catalytic domain expressed on the surface of T7 bacteriophage; and (3) a candidate PAK inhibitor substance (“test substance”) in solution in a series of known concentrations. When these three components are combined, the test substance is tested for its ability to compete, in a concentration-dependent manner, with the immobilized kinase bait probe for binding for binding to the phage-PAK catalytic domain. Afterwards, the amount of bait probe-bound phage-PAK can be detected, for example, by a phage plaque assay and/or quantitative PCR of phage DNA. The amount of probe-bound phage-PAK is inversely proportional to the affinity of the candidate inhibitor for the kinase and can be used in determining a Kd value of the test substance for the PAK catalytic site, as described below. The assay is described in further detail in Fabian et al. (2005, Nat. Biotech., 23:329) and Carter et al. (2005, Proc. Natl. Acad. Sci., USA, 102:11011); both of which are incorporated herein by reference.

Materials and Methods

Preparation of Kinase Fusion Constructs and of Phage Expressing Kinase Fusion Constructs

Various PAK isoforms and/or their catalytic domains were cloned in a modified version of the commercially available T7 Select 10-3 strain (Novagen). The head portion of each phage particle included 415 copies of the major capsid protein, and kinase fusion proteins are in approximately one to ten of these. Fusion proteins are randomly distributed across the phage head surface. The N terminus of the kinase is fused to the C terminus of the capsid protein through a flexible peptide linker. Kinases are linked to the T7 phage particle but are not incorporated into the phage head. Fusion proteins are randomly incorporated, and therefore distributed across the phage head surface. Clones of each kinase were sequenced, compared to an appropriate reference sequence, changed by site-directed mutagenesis where necessary to exactly match the reference sequence throughout the kinase domain, and transferred into the phage vector.

Kinase Assays

T7 kinase-tagged phage strains were grown in parallel in 24- or 96-well blocks in an E. coli host derived from the BL21 strain. E. coli were grown to log phase and infected with T7 phage from a frozen stock (multiplicity of infection ˜0.1) and incubated with shaking at 32° C. until lysis (approximately 90 minutes). Lysates were centrifuged (6,000× g) and filtered (0.2 μm) to remove cell debris. Streptavidin-coated magnetic beads were treated with staurosporine for 30 minutes at 25° C. to generate affinity resins for kinase assays. Liganded beads were blocked with excess biotin and washed with blocking buffer (SeaBlock [Pierce], 1% BSA, 0.05% Tween-20, 1 mM DTT) to remove unbound ligand and to reduce nonspecific phage binding. Binding reactions were assembled by combining phage lysates, liganded affinity beads and test compounds in 1× binding buffer (20% SeaBlock, 0.17×PBS, 0.05% Tween-20, 5 mM DTT). Test substances were prepared as 1,000× stocks in DMSO and rapidly diluted into the aqueous environment (0.1% DMSO final). DMSO (0.1%) was added to control assays lacking a test substance. All reactions were carried out in polystyrene 96-well plates that had been pretreated with blocking buffer in final volume of 0.1 ml. Assay plates were incubated at 25° C. with shaking for 1 hour, (e.g. long enough for binding reactions to reach equilibrium) and affinity beads were washed four times with wash buffer (1×PBS, 0.05% Tween-20, 1 mM DTT) to remove unbound phage. After the final wash, beads were resuspended in elution buffer (1×PBS, 0.05% Tween-20, 2 μM nonbiotinylated affinity ligand) and incubated at 25° C. with shaking for 30 minutes. Phage titer in the eluates was measured by standard plaque assays or by quantitative PCR.

Equilibrium binding equations yield the following expression for the binding constant for the interaction between the free test substance and the kinase (Kd (test)), assuming that the phage concentration is below Kd (test):

$${K}_{d} ( test ) = ( {K}_{d} ( probe ) / ( {K}_{d} ( probe ) + [ Probe ] ) ) ) * [ test ] 1 / 2$$

K_{d} (probe) is the binding constant for the interaction between the kinase and the immobilized ligand, [Probe] is the concentration of the immobilized ligand [test] ½ is the concentration of the free candidate compound at the midpoint of the transition. If [Probe] is below K_{d} (probe) the expression simplifies to K_{d} (test)=[test] ½. Under these conditions, binding constants measured for the interaction between kinases and candidate compounds (K_{d} (test)) are therefore independent of the affinity of the immobilized ligand for the kinase (K_{d} (probe)). T7 phage grow to a titer of 108-1010 plaque forming units (PFU)/ml, and the concentration of phage-tagged kinase in the binding reaction is therefore in the low pM range. The concentration of the immobilized ligand is kept in the low nM range, below its binding constant for the kinase. Binding data were fit to the equation:

PFU=L+((H−L)*(K_{d}(test)/(K_{d}(test)+[candidate])))

where L is the lower baseline, H is the upper baseline, K_{d} (test) is the binding constant for the interaction between the test substance and the kinase, and [test] is the free test substance concentration. Binding constants measured in duplicate on the same day as part of the same experiment generally were within twofold. Duplicate measurements performed on separate days generally varied by no more than fourfold. For kinase/test substance combinations where no interaction was observed, the binding constant was arbitrarily set to 1 M. K_{d} values were converted to p K_{d} (−log K_{d}), and clustering was based on the Pearson correlation. For further details of the assay system, see, for example, Fabian et al. (2005, Nat. Biotech., 23:329) and Carter et al. (2005, Proc. Natl. Acad. Sci., USA, 102:11011); both of which are incorporated herein by reference.

Using the above-described assay system, the interaction of a panel of 38 small molecule test substances was tested with the active sites of six PAK kinases: PAK1, PAK2, PAK3, PAK4, PAK6, and PAK7. As shown in Table 1, several compounds were found to have a K_{d} value less than 10 μM for one or more of the tested PAK isoforms.

TABLE 1
Results of Test PAK Inhibitor Affinity Assay

| | BMS- | | | | | | | | |
|---|---|---|---|---|---|---|---|---|---|
| | 387032/ | CHI4-258/ | | JNJ- | | Stauro- | | | VX-680/ |
| Kinase | SNS-032 | TKI-258 | EKB-569 | 7706621 | PKC-412 | sporine | SU-14813 | Sunitinib | MK-0457 |
| PAK1 | ≧10 μM | ≧10 μM | <5 μM | ≧10 μM | <5 μM | <1 μM | ≧10 μM | ≧10 μM | <5 μM |
| PAK2 | ≧10 μM | ≧10 μM | ≧10 μM | ≧10 μM | <5 μM | <1 μM | ≧10 μM | ≧10 μM | <10 μM |
| PAK3 | <5 μM | <1 μM | ≧10 μM | <5 μM | <1 μM | <1 μM | <1 μM | <0.2 μM | <5 μM |
| PAK4 | ≧10 μM | ≧10 μM | ≧10 μM | <5 μM | ≧10 μM | <1 μM | ≧10 μM | <5 μM | <5 μM |
| PAK6 | ≧10 μM | ≧10 μM | ≧10 μM | <5 μM | ≧10 μM | <1 μM | ≧10 μM | <5 μM | ≧10 μM |
| PAK7 | ≧10 μM | ≧10 μM | ≧10 μM | <1 μM | ≧10 μM | <1 μM | ≧10 μM | <1 μM | <5 μM |

Based on these data, specific compounds have been identified that have relatively high affinity for the catalytic domain of at least one PAK isoform, and are therefore useful inhibitors, as described herein. In some embodiments, such PAK inhibitors are used for the treatment and/or prophylaxis of mental disorders, e.g., Fragile X syndrome and autism spectrum disorders in animal models (e.g., FMR1 KO mice), as described herein, and in human clinical trials, as described herein.

### EXAMPLE 6

### Clinical Trial with EKB-569 or EKB-569 Derivative Compound

### Patient Selection

Patients (male or female) are between the ages of 14 and 40, have a verbal IQ≧60, and have a confirmed genetic diagnosis of fragile X syndrome. Patients have no known hypersensitivity to drugs. All studies are performed with institutional ethics committee approval and patient consent and/or parental permission (or a legal guardian's permission).

### Study Design

Study 1

Patients (n=15-20) are administered the EKB-569 and/or an EKB-569 derivative orally once daily for 30 days. Escalating doses of EKB-569 and/or an EKB-569 derivative between 0.1 mg/kg and 1 mg/kg are administered to cohorts of 3-6 patients until the maximum tolerated dose (MTD) is determined. The MTD is defined as the dose preceding that at which 2 of 3 or 2 of 6 patients experience dose-limiting toxicity.

Study 2

This is a randomized, double blind study. The study length is 42 days. Patients (n=60-80) are randomized to a control (placebo) or treatment group (once per day oral administration of EKB-569 MTD determined from previous study). Patients are scored on IQ tests, working memory tests, learning tests, and attention tests at the beginning of the study (i.e., prior to administration of control or EKB-569 treatments), at day 21, and at day 42. At the end of the study, patients are assessed for a significant increase in scores for each of the foregoing behavioral tests over the course of the study. A significant improvement in performance on any of the behavioral tests by patients treated with EKB-569 or an EKB-569 is considered a positive outcome for the use of EKB-569 in the treatment of fragile X syndrome.

### Equivalents and Scope

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. For example, it is to be understood that any of the compositions of the invention can be used for inhibiting the formation, progression, and/or recurrence of adhesions at any of the locations, and/or due to any of the causes discussed herein or known in the art. It is also to be understood that any of the compositions made according to the methods for preparing compositions disclosed herein can be used for inhibiting the formation, progression, and/or recurrence of adhesions at any of the locations, and/or due to any of the causes discussed herein or known in the art. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention (e.g., any small molecule PAK inhibitor, any biological activity of small molecule PAK inhibitors, any method of treatment of FXS and/or other neurodevelopmental disorder, any neurodevelopmental disorder, etc.), can be excluded from any one or more claims. For purposes of brevity, all of the embodiments in which one or more elements, features, purposes, or aspects is excluded are not set forth explicitly herein.

We claim:

1. A method, comprising steps of:
providing a subject susceptible to, suffering from, or exhibiting at least one symptom associated with fragile X syndrome (FXS); and
administering an amount of BMS-387032, SNS-032, CHI4-258, TKI-258, EKB-569, JNJ-7706621, PKC-412, staurosporine, SU-14813, sunitinib, VX-680, or MK-0457 to the subject effective to treat, alleviate, ameliorate, relieve, delay onset of, inhibit progression of, reduce severity of, or reduce incidence of the at least one symptom associated with FXS.

2. A method, comprising steps of:
providing a subject susceptible to, suffering from, or exhibiting at least one symptom associated with fragile X syndrome (FXS); and
administering an amount of a pharmaceutical composition comprising BMS-387032, SNS-032, CHI4-258, TKI-258, EKB-569, JNJ-7706621, PKC-412, staurosporine, SU-14813, sunitinib, VX-680, or MK-0457 to the subject effective to treat, alleviate, ameliorate, relieve, delay onset of, inhibit progression of, reduce severity of, or reduce incidence of the at least one symptom associated with FXS.

3. A method, comprising steps of:
providing a subject susceptible to, suffering from, or exhibiting at least one symptom associated with mental retardation or autism spectrum disorders; and
administering an amount of BMS-387032, SNS-032, CHI4-258, TKI-258, EKB-569, JNJ-7706621, PKC-412, staurosporine, SU-14813, sunitinib, VX-680, or MK-0457 to the subject effective to treat, alleviate, ameliorate, relieve, delay onset of, inhibit progression of, reduce severity of, or reduce incidence of the at least one symptom associated with mental retardation or autism spectrum disorders.

4. A method, comprising steps of:
providing a subject susceptible to, suffering from, or exhibiting at least one symptom associated with mental retardation or autism spectrum disorders; and
administering an amount of a pharmaceutical composition comprising BMS-387032, SNS-032, CHI4-258, TKI-258, EKB-569, JNJ-7706621, PKC-412, staurosporine, SU-14813, sunitinib, VX-680, or MK-0457 to the subject effective to treat, alleviate, ameliorate, relieve, delay onset of, inhibit progression of, reduce severity of, or reduce incidence of the at least one symptom associated with mental retardation or autism spectrum disorders.

5. The method of any one of claims 1-4, wherein the small molecular inhibitor is selective for a PAK selected from the group consisting of PAK1, PAK2, PAK3, PAK4, PAK6, and PAK7.

6. A kit comprising:
a pharmaceutical composition comprising BMS-387032, SNS-032, CHI4-258, TKI-258, EKB-569, JNJ-7706621, PKC-412, staurosporine, SU-14813, sunitinib, VX-680, or MK-0457;
instructions for administering the pharmaceutical composition to a subject suffering from, susceptible to, or exhibiting symptoms of FXS.

7. A kit comprising:
a pharmaceutical composition comprising BMS-387032, SNS-032, CHI4-258, TKI-258, EKB-569, JNJ-7706621, PKC-412, staurosporine, SU-14813, sunitinib, VX-680, or MK-0457;
instructions for administering the pharmaceutical composition to a subject suffering from, susceptible to, or exhibiting symptoms of mental retardation or autism spectrum disorders.


SEQUENCE LISTING (18 sequences)

SEQ ID NO: 1 - protein, 681 aa, Artificial Sequence
  Drosophila melanogaster FMR1
MEDLLVEVRLDNGAYYKGQVTAVADDGIFVDVDGVPESMKYPFVNVRLPPEETVEVAAPIFEEGMEVEVFTRTNDRETCGWWVGIIKMRKAEIYAVAYIGFETSYTEICELGRLRAKNSNPPITAKTFYQFTLPVPEELREEAQKDGIHKEFQRTIDAGVCNYSRDLDALIVISKFEHTQKRASMLKDMHFRNLSQKVMLLKRTEEAARQLETTKLMSRGNYVEEFRVRDDLMGLAIGSHGSNIQAARTVDGVTNIELEEKSCTFKISGETEESVQRARAMLEYAEEFFQVPRELVGKVIGKNGRIIQEIVDKSGVFRIKIAGDDEQDQNIPRELAHVPLVFIGTVESIANAKVLLLYHLSHLKEVEQLRQEKMEIDQQLRAIQESSMGSTQSFPVTRRSERGYSSDIESVRSMRGGGGGQRGRVRGRGGGGPGGGNGLNQRYHNNRRDEDDYNSRGDHQRDQQRGYNDRGGGDNTGSYRGGGGGAGGPGNNRRGGINRRPPRNDQQNGRDYQHHNHTTEEVRETREMSSVERADSNSSYEGSSRRRRRQKNNNGPSNTNGAVANNNNKPQSAQQPQQQQPPAPGNKAALNASDASKQNSGNANAAGGASKPKDASRNGDKQQAGTQQQQPSQVQQQQAAQQQQPKPRRNKNRSNNHTDQPSQQQQLAENVKKEGLVNGTS

SEQ ID NO: 2 - protein, 632 aa, Artificial Sequence
  Human FMR1
MEELVVEVRGSNFAFYKAFVKDVHEDSITVAFENNWQPDRQIPFHDVRFPPPVGYNKGINESDEVEVYSRANEKEPCCWWLAKVRMIKGEFYVIEYAACDATYNEIVTIERLRSVNPNKPATKDTEFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAILDEDTCTFHIYGEDQDAVKKARSFLEFAEDYIQVPRNLVGKVIGKNGLKIQEIVDKSGVVRVRIEAENEKNVPQEEEIMPPNSLPSNNSRVGPNAPEEKKHLDIKENSTHFSQPNSTKVQRVLVASSVVAGESQKPELKAWQGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVP

SEQ ID NO: 3 - protein, 621 aa, Artificial Sequence
  Human FXR1
MADVTVEVRGSNGAFYKGFIKDVHEDSLTVVFENNWQPERQVPFNEVRLPPPPDIKKEISEGDEVEVYSRANDQEPCGWWLAKVRMMKGEFYVIEYAACDATYNEIVTFERLRPVNQNKTVKKNTFFKCTVDVPEDLREACANENAHKDFKKAVGACRIFYHPETTQLMILSASEATVKRVNILSDMHLRSIRTKLMLMSRNEEATKHLECTKQLAAAFHEEFVVREDLMGLAIGTHGSNIQQARKVPGVTAIELDEDTGTFRIYGESADAVKKARGFLEFVEDFIQVPRELVGKVIGKNGRVIQEIVDKSGVVRIRIEGDNENKLPREDGMVPFVFVGTKESIGNVQVLLEYHIAYLKEVEQLRMERLQIDEQLRQIGSRSYSGRGRGRRGPNYTSGYGTNSELSNPSETESERKDELSDWSLAGEDNRDSRHQRDSRRRPGGRGRSVSGGRGRGGPRGRKSSISSVLKDPDSNPYSLLDNTESDQTADTDASESHHSTNRRRRSRRRRTDEDAVLMDGMTESDTASVNENGLVTVADYISRAESQSRQRNLPRETLAKNKKEMAKDVIELHGPSEKAINGPTSASGDDISKLQRTPGEEKINTLKEENTQEAAVLNGVS

SEQ ID NO: 4 - protein, 673 aa, Artificial Sequence
  Human FXR2
MGGLASGGDVEPGLPVEVRGSNGAFYKGFVKDVHEDSVTIFFENNWQSERQIPFGDVRLPPPADYNKEITEGDEVEVYSRANEQEPCGWWLARVRMMKGDFYVIEYAACDATYNEIVTLERLRPVNPNPLATKGSFFKVTMAVPEDLREACSNENVHKEFKKALGANCIFLNITNSELFILSTTEAPVKRASLLGDMHFRSLRTKLLLMSRNEEATKHLETSKQLAAAFQEEFTVREDLMGLAIGTHGANIQQARKVPGVTAIELGEETCTFRIYGETPEACRQARSYLEFSEDSVQVPRELVGKVIGKNGRVIQEIVDKSGVVRVRVEGDNDKKNPREEGMVPFIFVGTRENISNAQALLLYHLSYLQEVEQLRLERLQIDEQLRQIQLGFRPPGSGRGSGGSDKAGYSTDESSSSSLHATRTYGGSYGGRGRGRRTGGPAYGPSSDVSTASETESEKRELPNRAGPGDRDPPTRGEESRRRPTGGRGRGPPPAPRPTSRYNSSSISSVLKDPDSNPYSLLDTSEPEPPVDSEPGEPPPASARRRRSRRRRTDEDRTVMDGGLESDGPNMTENGLEDESRPQRRNRSRRRRNRGNRTDGSISGDRQPVTVADYISRAESQSRQSAPLERTKPSLDSLSGQKGDSVSKLPKGPSENGELSAPLELGSMVNGVS

SEQ ID NO: 5 - protein, 614 aa, Artificial Sequence
  Mouse FMR1
MEELVVEVRGSNGAFYKAFVKDVHEDSITVAFENNWQPERQIPFHDVRFPPPVGYNKDINESDEVEVYSRANEKEPCCWWLAKVRMIKGEFYVIEYAACDATYNEIVTIERLRSVNPNKPATKDTFHKIKLEVPEDLRQMCAKESAHKDFKKAVGAFSVTYDPENYQLVILSINEVTSKRAHMLIDMHFRSLRTKLSLILRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKSVPQEEEIMPPSSLPSNNSRVGPNSSEEKKHLDTKENTHFSQPNSTKVQRVLVVSSIVAGGPQKPEPKAWQGMVPFVFVGTKDSIANATVLLDYHLNYLKEVDQLRLERLQIDEQLRQIGASSRPPPNRTDKEKGYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTADGSLQSASSEGSRLRTGKDRNQKKEKPDSVDGLQPLVNGVP

SEQ ID NO: 6 - protein, 545 aa, Artificial Sequence
  Human PAK1
MSNNGLDIQDKPPAPPMRNTSTMIGVGSKDAGTLNHGSKPLPPNPRRKKKKDRFYRSILPGDKTNKKKHKERPRISLPSDFEHTIHVGFDAVTGEFTGMPEQMARLLQTSNITKSEQKKNPQAVLDVLEFYNSKKTSNSQKYMSFTDKSAEDYNSSNALNVKAVSETPAVPPVSRDEDDDDDDATPPPVIAPRPEHTKSVYTRSVIRPLPVTPTRDVATSPISPTENNTTPPDALTRNTEKQKKKPKMSDEEILRKLRSIVSVGDPKKKYTRFEKDGQGASGTVYTAMDVATGQEVAIKQMNLQQQPKKELIINRILVMRENKNPNIVNYLDSYLVGDELMVVMEYLAGGSLTDVVTETCMDEGQIAAVCRECLQALEFLHSNQVIHRDIKSDNILLGMDGSVKLTDFGFCAQITPEQSKRSTMVGTPYMMAPEVVTRKAYGPKVDIMSLGIMAIEMIEGEPPYLNENPLRALYLIATNGTPELQNPEKLSAIFRDFLNRCLDMDVEKRGSAKELLQHQFLKIAKPLSSLTPLIAAAKEATKNNH

SEQ ID NO: 7 - protein, 525 aa, Artificial Sequence
  Human PAK2
MSDNGELRDKPPAPPVRMSSTIFSTGGKDPLSANHSLKPLPSVPRRKKPRHKIISIFSGTEKGSKKKEKERPRISPPSDFEHTIHVGFDAVTGEFTGMPEQMARLLQTSNITKLEQKKNPQAVLDVLKFYDSNTVKQKYLSFTPPEKDGLPSGTPALNAKGTEAPAVVTFEEDDDEETAPPVIAPRPDHTKSIYTRSVIDPVPAPVGDSHVDCAAKSLDKQKKKPKMTDEEIMRKLRTIVSIGNPKKKYTRYEKDGQGASGTVFTATDVALGQEVAIKQINLQKQPKKELIINRILVMKELKNPNIVNFLDSYLVGDELFVVMEYLAGGSLTDVVTETACMDEAQIAAVCRECLQALEFLHANQVIHRDIKSDNVLLGMEGSVKLTDFGFCAQITPEQSKRSTMVGTPYMMAPEVVTRKAYGPKVDIMSLGIMAIEMVEGEPPYLNENPLRALYLIATNGTPELQNPEKLSPIFRDFLNRCLEMDVEKRGSAKELLQHPFLKLAKPLSSLTPLIMAAKEAMKSNR

SEQ ID NO: 8 - protein, 544 aa, Artificial Sequence
  Human PAK3
MSDGLDNEEKPPAPPLRMNSNNRDSSALNHSSKPLPMAPSEKNKKARLRSIFPGGGDKTNKKKEKERPRISLPSDFEHTIHVGFDAVTGEFTGIPEQMARLLQTSNITKLEQKKNPQAVLDVLKFYDSKETVNNQKYMSFTSGDKSAHGYIAAHPSSTKTASEPPLAPPVSRRRDRRRRRRRDRNEPPPVIAPRPEHTKSIYTRSVVESIASPAVPNKEVTPPSAENANSSTLYRNTDRQRKKSKNTDEEILRKLRSIVSVGDPKKKYTRERKDGQGASGTVYTALDIATGQEVAIKQMNLQQQPKKELIINRILVMRENKNPNIVNYLDSYLVGDELMVVMEYLAGGSLTDVVTETCMDEGQIAAVCRECLQALDFLHSNQVIHRDIKSDNILLGMDGSVKLTDFGFCAQITPEQSKRSTMVGTPYMMAPEVVTRKAYGPKVDIMSLGIMAIEMVEGEPPYLNENPLRALYLIATNGTPELQNPERLSAVPRDFLNRCLEMDVERRGSAKELLQHFPLKLAKPLSSLTPLIIAAKEAIKNSSR

SEQ ID NO: 9 - protein, 591 aa, Artificial Sequence
  Human PAK4
MFGKRKKRVEISAPSNFEHRVHTGFDQHEQKFTGLPRQWQSLIEESARRPKPLVDPACITSIQPGAPKTIVRGSKGAKDGALTLLLDEFENMSVTRSNSLRRDSPPPPARARQENGMPEEPATTARGGPGKAGSRGRFAGHSEAGGGSGDRRRAGPEKRPKSSREGSGGPQESSRDKRPLSGPDVGTPQPAGLASGAKLAAGRPFNTYPRADTDHPSRGAQGEPHDVAPNGPSAGGLAIPQSSSSSSRPPTRARGAPSPGVLGPHASEPQLAPPACTPAAPAVPGPPGPRSPQREPQRVSHEQFRAALQLVVDPGDPRSYLDNFIKIGEGSTGIVCIATVRSSGKLVAVKKMDLRKQQRRELLFNEVVIMRDYQHENVVEMYNSYLVGDELWVVMEFLEGGALTDIVTHTRMNEEQIAAVCLAVLQALSVLHAQGVIHRDIKSDSILLTHDGRVKLSDFGFCAQVSKEVPRRKSLVGTPYWMAPELISRLPYGPEVDIWSLGIMVIEWVDGEPPYFNEPPLKAMKMIRDNLPPRLKNLHKVSPSLKGFLDRLLVRDPAQRATAAELLKHPFLAKAGPPASIVPLMRQNRTR

SEQ ID NO: 10 - protein, 719 aa, Artificial Sequence
  Human PAK7
MFGKKKKKIEISGPSNFEHRVHTGFDPQEQKFTGLPQQWHSLLADTANRPKPMVDPSCITPIQLAPMKTIVRGNKPCKETSINGLLEDFDNISVTRSNSLRKESPPTPDQGASSHGPGHAEENGFITFSQYSSESDTTADYTTEKYREKSLYGDDLDPYYRGSHAAKQNGHVMKMKHGEAYYSEVKPLKSDFARFSADYHSHLDSLSKPSEYSDLKWEYQRASSSSPLDYSFQFTPSRTAGTSGCSKESLAYSESEWGPSLDDYDRRPKSSYLNQTSPQPTMRQRSRSGSGLQEPMMPFGASAFKTHPQGHSYNSYTYPRLSEPTMCIPKVDYDRAQMVLSPPLSGSDTYPRGPAKLPQSQSKSGYSSSSHQYPSGYHKATLYHHPSLQSSSQYISTASYLSSLSLSSSTYPPPSWGSSSDQQPSRVSHEQFRAALQLVVSPGDPREYLANFIKIGEGSTGIVCIATEKHTGKQVAVKKMDLRKQQRRELLFNEVVIMRDYHHDNVVDMYSSYLVGDELWVVMEFLEGGALTDIVTHTRMNEEQIATVCLSVLRALSYLHNQGVIHRDIKSDSILLTSDGRIKLSDFGFCAQVSKEVPKRKSLVGTPYWMAPEVISRLPYGTEVDIWSLGIMVIEMIDGEPPYFNEPPLQAMRRIRDSLPPRVKDLHKVSSVLRGFLDLMLVREPSQRATAQELLGHPFLKLAGPPSCIVPLMRQYRHH

SEQ ID NO: 11 - protein, 681 aa, Artificial Sequence
  Human PAK5
MFRKKKKKRPEISAPQNFQHRVHTSFDPKEGKFVGLPPQWQNILDTLRRPKPVVDPSRITRVQLQPMKTVVRGSAMPVDGYISGLLNDIQKLSVISSNTLRGRSPTSRRRAQSLGLLGDEHWATDPDMYLQSPQSERTDPHGLYLSCNGGTPAGHKQMPQPEPQSPRVLPNGLAAKQSLGPAEFQGASQRCLQLGACLQSSPPGASPPTGTNRHGMKAAKHGSEEARPQSCLVGSATGRPGGEGSPSPKTRESSLKRRLFRSMFLSTAATAPPSSSKPGPPPQSKPNSSFRPPQKDNPPSLVAKAQSLPSDQPVGTFSPLTTSDTSSPQKSLRTAPATGQLPGRSSPAGSPRTWHAQISTSNLYLPQDPTVAKGALAGEDTGVVTHEQFKAALRMVVDQGDPRLLLDSYVKIGEGSTGIVCLAREKHSGRQVAVKMMDLRKQQRRELLFNEVVIMRDYQHFNVVEMYKSYLVGEELWVLMEFLQGGALTDIVSQVRLNEEQIATVCEAVLQALAYLHAQGVIHRDIKSDSILLTLDGRVKLSDFGFCAQISKDVPKRKSLVGTPYWWMAPEVISRSLYATEVDIWSLGIMVIEMVDGEPPYFSDSPVQAMKRLRDSPPPKLKNSHKVSPVLRDFLERMLVRDPQERATAQELLDHPFLLQTGLPECLVPLIGLYRKQTSTC

SEQ ID NO: 12 - protein, 681 aa, Artificial Sequence
  Human PAK6
MFRKKKKKRPEISAPQNFQHRVHTSFDPKEGKFVGLPPQWQNILDTLRRPKPVVDPSRITRVQLQPMKTVVRGSAMPVDGYISGLLNDIQKLSVISSNTLRGRSPTSRRRAQSLGLLGDEHWATDPDMYLQSPQSERTDPHGLYLSCNGGTPAGHKQMPWPEPQSPRVLPNGLAAKAQSLGPAEFQGASQRCLQLGACLQSSPPGASPPTGTNRHGMKAAKHGSEEARPQSCLVGSATGRPGGEGSPSPKTRESSLKRRLFRSMFLSTAATAPPSSSKPGPPPQSKPNSSFRPPQKDNPPSLVAKAQSLPSDQPVGTFSPLTTSDTSSPQKSLRTAPATGQLPGRSSPAGSPRTWHAQISTSNLYLPQDPTVAKGALAGEDTGVVTHEQFKAALRMVVDQGDPRLLLDSYVKIGEGSTGIVCLAREKHSGRQVAVKMMDLRKQQRRELLFNEVVIMRDYQHFNVVEMYKSYLVGEELWVLMEFLQGGALTDIVSQVRLNEEQIATVCEAVLQALAYLHAQGVIHRDIKDSDILLTLDGRVKLSDFGFCAQISKDVPKRKSLVGTPYWMAPEVISRSLYATEVDIWSLGIMVIEMVDGEPPYFSDSPVQAMKRLRDSPPPKLKNSHKVSPVLRDFLERMLVRDPQERATAQELLDHPFLLQTGLPECLVPLIQLYRKQTSTC

SEQ ID NO: 13 - protein, 719 aa, Artificial Sequence
  Human PAK4
MFGKKKKKIEISGPSNFEHRVHTGFDPQEQKFTGLPQQWHSLLADTANRPKPMVDPSCITPIQLAPMKTIVRGNKPCKETSINGLLEDFDNISVTRSNSLRKESPPTPDQGASSHGPGHAEENGFITFSQYSSESDTTADYTTEKYREKSLYGDDLDPYYRGSHAAKQNGHVMKMKHGEAYYSEVKPLKSDFARFSADYHSHLDSLSKPSEYSDLKWEYQRASSSSPLDYSFQFTPSRTAGTSGCSKESLAYSESEWGPSLDDYDRRPKSSYLNQTSPQPTMRQRSRSGSGLQEPMMPFGASAFKTHPQGHSYNSYTYPRLSEPTMCIPKVDYDRAQMVLSPPLSGSDTYPRGPAKLPQSQSKSGYSSSSHQYPSGYHKATLYHHPSLQSSSQYISTASYLSSLSLSSSTYPPPSWGSSSDQQPSRVSHEQFRAALQLVVSPGDPREYLANFIKIGEGSTGIVCIATEKHTGKQVAVKKMDLRKQQRRELLFNEVVIMRDYHHDNVVDMYSSYLVGDELWVVMEFLEGGALTDIVTHTRMNEEQIATVCLSVLRALSYLHNQGVIHRDIKSDSILLTSDGRIKLSDEGFCAQVSKEVPKRKSLVGTPYWMAPEVISRLPYGTEVDIWSLGIMVIEMIDGEPPYFNEPPLQAMRRIRDSLPPRVKDLHKVSSVLRGFLDLMLVREPSQRATAQELLGHPFLKLAGPPSCIVPLMRQYRHH

SEQ ID NO: 14 - protein, 591 aa, Artificial Sequence
  Human PAK5
MFGKRKKRVEISAPSNFEHRVHTGFDQHEQKFTGLPRQWQSLIEESARRPKPLVDPACITSIQPGAPKTIVRGSKGAKDGALTLLLDEFENMSVTRSNSLRRDSPPPPARARQENGMPEEPATTARGGPGKAGSRGRFAGHSEAGGGSGDRRRAGPEKRPKSSREGSGGPQESSRDKRPLSGPDVGTPQPAGLASGAKLAAGRPFNTYPRADTDHPSRGAQGEPHDVAPNGPSAGGLAIPQSSSSSSRPPTRARGAPSPGVLGPHASEPQLAPPACTPAAPAVPGPPGPRSPQREPQRVSHEQFRAALQLVVDPGDPRSYLDNFIKIGEGSTGIVCIATVRSSGKLVAVKKMDLRKQQRRELLFNEVVIMRDYQHENVVEMYNSYLVGDELWVVMEFLEGGALTDIVTHTRMNEEQIAAVCLAVLQALSVLHAQGVIHRDIKSDSILLTHDGRVKLSDFGFCAQVSKEVPRRKSLVGTPYWMAPELISRLPYGPEVDIWSLGIMVIEMVDGEPPYFNEPPLKAMKMIRDNLPPRLKNLHKVSPSLKGFLDRLLVRDPAQRATAAELLKHPFLAKAGPPASIVPLMRQNRTR

SEQ ID NO: 15 - protein, 681 aa, Artificial Sequence
  Human PAK6
MFRKKKKKRPEISAPQNFQHRVHTSFDPKEGKFVGLPPQWQNILDTLRRPKPVVDPSRITRVQLQPMKTVVRGSAMPVDGYISGLLNDIQKLSVISSNTLRGRSPTSRRRAQSLGLLGDEHWATDPDMYLQSPQSERTDPHGLYLSCNGGTPAGHKQMPWPEPQSPRVLPNGLAAKAQSLGPAEFQGASQRCLQLGACLQSSPPGASPPTGTNRHGMKAAKHGSEEARPQSCLVGSATGRPGGEGSPSPKTRESSLKRRLFRSMFLSTAATAPPSSSKPGPPPQSKPNSSFRPPQKDNPPSLVAKAQSLPSDQPVGTFSPLTTSDTSSPQKSLRTAPATGQLPGRSSPAGSPRTWHAQISTSNLYLPQDPTVAKGALAGEDTGVVTHEQFKAALRMVVDQGDPRLLLDSYVKIGEGSTGIVCLAREKHSGRQVAVKMMDLRKQQRRELLFNEVVIMRDYQHFNVVEMYKSYLVGEELWVLMEFLQGGALTDIVSQVRLNEEQIATVCEAVLQALAYLHAQGVIHRDIKSDSILLTLDGRVKLSDFGFCAQISKDVPKRKSLVGTPYWMAPEVISRSLYATEVDIWSLGIMVIEMVDGEPPYFSDSPVQAMKRLRDSPPPKLKNSHKVSPVLRDFLERMLVRDPQERATAQELLDHPFLLQTGLPECLVPLIQLYRKQTSTC

SEQ ID NO: 16 - protein, 572 aa, Artificial Sequence
  Caenorhabditis elegans PAK1
MKAFSSYDEKPPAPPIRFSSSATRENQVVGLKPLPKEPEATKKKKTMPNPFMKKNKDKKEASEKPVISRPSNFEHTIHVGYDPKTGEFTGMPEAWARLLTDSQISKQEQQQNPQAVLDALKYYTQGESSGQKWLQYDMMFIDDAPSRTPSYGLKPQPYSTSSLPYHGNKIQDPRKMNPMTTSTSSAGYNSKQGVPPTTFSVNENRSSMPPSYAPPPVPHGETPADIVPPAIPDRPARTLSIYTKPKEEEEKIPDLSKGAQGVQARGQKAKKKMTDAEVLTKLRTIVSIGNPDRKYRKVDKIGSGASGSVYTAIEISTEAEVAIKQMNLKDQPKKELIINEILVMRENKHANIVNYLDSYLVCDELWVVMEYLAGGSLTDVVTECQMEDGIIAAVCREVLQALEFLHSRHVIHRDIKSDNILLGMDGSVKLTDFGFCAQLSPEQRKRTTMVGTPYWMAPEVVTRKQYGPKVDVWSLGIMAIEMVEGEPPYLNENPLRAIYLIATNGKPDFPGRDSMTLLFKDFVDSALEVQVENRWSASQLLTHPFLRCAKPLASLYYLIVAAKKSIAEASNS

SEQ ID NO: 17 - protein, 704 aa, Artificial Sequence
  Drosophila melanogaster PAK1
MSSEEDKPPAPPVRLTSNRGGNERSGGGVGVGGGGLGGGGMGDVPPDMRPLPKEPDDSDRKKKTLKSKIKGSKPSHTDSKPNISYPTNFEHTVHVGFDAVTGEFTGMPEAWARLLMNSNISKQEQKKNPQAVLDVLKWFDNTTKQRPSSKYMTNAITTHSGSSLSRVSSSSPSSTPTDSELHGSNSGGNLIGVQLGSMTLGPNANNVAVAGILGNHYQQQQQHLLQQQQPLLHQNHNQHHMGISQSHSYNFVGHTVSSSTSQHSSANEDDMLGPQHPQQQPPPPPVASRPERTKSIYTRPIEDLQPAIIPMPVAPATTPATPLQNHRTPGGISAPATSPMMHNNATTTLDKNKNNANLYTPEPTVAQVSAGGPSSQVAGNQIAVPQAAVAPAATPNTRAANAKKKKMSDEEILEKLRTIVSVGDPNRKYTKMEKIGQGASGTVYTAIESSTGMEVAIKQMNLSQQPKKELIINEILVMRENKHPNVVNYLDSYLVSEELWVVMEYLPGGSLTDVVTETCMDEGQIAAVCREVLQALEFLHANQVIHRDIKSDNILLGLDGSVKLTDFGFCAQISPEQSKRTTMVGTPYWMAPEVVTRKQYGPKVDLQWSLGIMAIEMVEGEPPYLNENPLKALYLIATNGKPEIKEKDKLSSAFQDFLDQCLEVEVDRRASALDLLKHPFLKLARPLASLTPLIMAAKEATKGN

SEQ ID NO: 18 - protein, 544 aa, Artificial Sequence
  Rat PAK1
MSNNGLDVQDKPPAPPMRNTSTMIGAGSKDPGTLNHGSKPLPPNPEEKKKKDRFYRSILAGDKTNKKKEKERHEISLPSDFEHTIHVGFDAVTGEFTGMPEQWARLLQTSNITKSEQKKNPQAVLDVLEFYNSKKTSNSQKYMSFTDKSAEDYNSSNTLNVKTVSETPAVPPVSEDEDDDDDATPPPVIAPRPEHTKSVYTRSVIEPLPVTPTRDVATSPISPTENNTTPPDALTRNTEKQKKKPKMSDEEILEKLRNIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQHQFLKIAKPLSSLTPLIAAAKEATKNNH